(19) United States
(12) Patent Application Publication
(10) Pub. No.: US 2006/0110784 A1
(43) Pub. Date: May 25, 2006

(54) Site-specific labeling of proteins for NMR studies

(75) Inventors: Alexander Deiters, Raleigh, NC (US); Bernhard H. Geierstanger, Del Mar, CA (US); Peter G. Schultz, La Jolla, CA (US)

Correspondence Address: QUINE INTELLECTUAL PROPERTY LAW GROUP, P.C., P O BOX 458, ALAMEDA, CA, 94501, US

(73) Assignees: The Scripps Research Institute, La Jolla, CA (US); IRM, LLC, Hamilton (BM)

(21) Appl. No.: 11/233,466

(22) Filed: Sep. 21, 2005

Related U.S. Application Data
(60) Provisional application No. 60/612,343, filed on Sep. 22, 2004.
(60) Provisional application No. 60/645,926, filed on Jan. 21, 2005.

(51) Int. Cl. C12Q 1/37; G01N 33/00; C12N 15/74; C12N 1/21; C12N 5/06; C12N 1/18

(52) U.S. Cl. 435/23; 435/483; 435/488; 435/252.33; 435/325; 435/254.2; 436/86

(57) ABSTRACT

Methods of producing and/or analyzing spectroscopically labeled proteins, e.g., proteins site-specifically labeled with NMR active isotopes, spin-labels, chelators for paramagnetic metals, and the like, are provided. The labeled proteins are produced in translation systems including orthogonal aminoacyl tRNA synthetase/tRNA pairs. Methods for assigning NMR resonances, e.g., methods using isotopically labeled proteins, are also provided.

### CROSS-REFERENCE TO RELATED APPLICATIONS

This application is related to U.S. provisional patent applications U.S. Ser. No. 60/612,343 filed Sep. 22, 2004 and U.S. Ser. No. 60/645,926 filed Jan. 21, 2005. The present application claims priority to, and benefit of, these applications, pursuant to 35 U.S.C. § 119(e) and any other applicable statute or rule. Each of these applications is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with government support under Grant GM62159 from the National Institutes of Health. The government may have certain rights to this invention.

### FIELD OF THE INVENTION

This invention is in the field of translation biochemistry. The invention relates to methods of producing and/or analyzing spectroscopically labeled proteins, e.g., proteins site-specifically labeled with NMR active isotopes, spin-labels, chelators for paramagnetic metals, and the like. The invention also relates to methods for assigning NMR resonances.

### BACKGROUND OF THE INVENTION

Studies of biological macromolecules by NMR (Nuclear Magnetic Resonance) spectroscopy become increasingly difficult as the molecular weight of the molecule of interest increases, due to signal overlap and signal reduction resulting from faster transverse relaxation. Partial and uniform ²H-, ¹³C-, and ¹⁵N-labeling of proteins combined with heteronuclear, multidimensional NMR experiments can overcome these problems to some extent and has allowed the elucidation of structures of proteins with a molecular weight of 30 kDa (Goto and Kay (2000) Curr. Opin. Struct. Biol. 10:585; Gardner (1998) Annu. Rev. Biophys. Biomol. Struct. 27:357; Wüthrich (2003) Angew. Chem. Int. Ed. 42:3340; and Bax (1994) Curr. Opin. Struct. Biol. 4:738). The development of transverse relaxation optimized spectroscopy (TROSY) has extended the limit of solution NMR studies to systems as large as 900 kDa (Pervushin et al. (1997) Proc. Natl. Acad. Sci. U.S.A. 94:12366; Fiaux et al. (2002) Nature 418:207; and Fernandez and Wider (2003) Curr. Opin. Struct. Biol. 13:570). Ultimately, however, the resonances in large proteins can become impossible to resolve even at the highest available magnetic fields.

Assignment of resonances to particular amino acids in a protein is a key step in NMR studies. Such assignments can be facilitated, e.g., in studies of larger proteins, by site-specific labeling of one or more amino acids with an NMR active isotope (see, e.g., Ellman et al. (1992) J. Am. Chem. Soc. 114:7959).

To obtain sufficient quantities for NMR measurements, most isotopically labeled proteins are recombinantly expressed in E. coli using minimal media in combination with ¹³C glucose, ¹⁵N ammonium salts, and deuterium oxide. However, such techniques typically label many, if not all, amino acid residues in the protein simultaneously. Strategies for more selective incorporations of isotopes include feeding experiments with labeled amino acids in defined media (Gardner (1998) Annu. Rev. Biophys. Biomol. Struct. 27:357), often utilizing auxotrophic bacterial expression strains, ‘reverse isotope’ labeling (Vuister et al. (1994) J. Am. Chem. Soc. 116:9206; Kelly et al. (1999) J. Biomol. NMR 14:79), segmental labeling by transsplicing (Yamazaki (1998) J. Am. Chem. Soc. 120:5591), or total and semi-synthesis by chemical ligation (Xu et al. (1999) Proc. Natl. Acad. Sci. USA 96:388) and cell-free expression systems using chemically aminoacylated suppressor tRNAs (Yabuki et al. (1998) J. Biomol. NMR 11:295). Although site-specific incorporation of isotopic labels into a protein has been demonstrated by the latter method (Ellman et al. (1992) J. Am. Chem. Soc. 114:7959), the production of milligram quantities sufficient for NMR measurements is tedious and expensive.

There is thus a need for methods that facilitate site-specific incorporation of isotopically labeled amino acids into proteins for NMR analysis. The present invention addresses these and other needs, as will be apparent upon review of the following disclosure.

### SUMMARY OF THE INVENTION

The present invention provides methods for producing and/or analyzing spectroscopically labeled proteins through site-specific incorporation of spectroscopically labeled unnatural amino acids into the proteins, using translation systems including orthogonal aminoacyl tRNA synthetases and orthogonal tRNAs. The invention also provides methods for assigning NMR resonances by site-specifically incorporating isotopically labeled unnatural amino acids into proteins using such translation systems. The invention also provides methods for producing and/or analyzing spectroscopically labeled proteins through site-specific incorporation of unnatural amino acids into the proteins, using translation systems including orthogonal aminoacyl tRNA synthetases and orthogonal tRNAs, followed by attachment of spectroscopic labels to the unnatural amino acids.

Thus, a first general class of embodiments provides methods for producing and/or analyzing a spectroscopically labeled protein. In the methods, a nucleic acid that encodes the protein is translated in a translation system. The nucleic acid includes a selector codon. The translation system includes an orthogonal tRNA (O-tRNA) that recognizes the selector codon, an unnatural amino acid comprising a spectroscopic label, and an orthogonal aminoacyl tRNA synthetase (O-RS) that preferentially aminoacylates the O-tRNA with the unnatural amino acid. The unnatural amino acid is incorporated into the protein as it is translated, thereby producing the spectroscopically labeled protein.

In one class of embodiments, the unnatural amino acid comprises a) an isotopically labeled unnatural amino acid comprising an NMR active isotope selected from the group consisting of: ⁷Li, ¹³B, ¹⁴N, ¹⁵N, ¹⁷O, ¹⁹F, ²³Na, ²⁷Al, ²⁹Si, ³¹P, ⁵⁹Co, ⁷⁷Se, ¹¹³Cd, ¹¹⁹Sn, ¹⁹⁵Pt, and a combination thereof, b) a spin-labeled amino acid, or c) a chelator for a paramagnetic metal, and the spectroscopically labeled protein is subjected to NMR spectroscopy.

In one class of embodiments, the unnatural amino acid comprises an isotopically labeled unnatural amino acid. For example, the isotopically labeled unnatural amino acid can include a radioactive isotope or, preferably, an NMR active isotope. The NMR active isotope is optionally selected from the group consisting of ²H, ³H, ¹³C, ¹⁵N, ⁷Li, ¹³B, ¹⁴N, ¹⁷O, ¹⁹F, ²³Na, ²⁷Al, ²⁹Si, ³¹P, ⁵⁹Co, ⁷⁷Se, ¹¹³Cd, ¹¹⁹Sn, and ¹⁹⁵Pt.

The NMR active (or other) isotope can be attached to or incorporated into the unnatural amino acid at essentially any convenient position. As just a few examples, the NMR active isotope can be part of a methyl group, an amino group, an azido group, a keto group, a carboxy group, a cyano group, an alkyl group, an alkoxy group, an alkynyl moiety, a thiol group, a halogen atom, an aryl group, a sugar residue, a photocrosslinking moiety, or a photolabile group.

Similarly, essentially any unnatural amino acid can be isotopically labeled. For example, the isotopically labeled unnatural amino acid can be O-methyl-L-tyrosine, e.g., in which the methyl group is isotopically labeled, or in which the nitrogen is isotopically labeled (i.e., the isotopically labeled unnatural amino acid can be ¹⁵N-labeled p-methoxyphenylalanine).

The protein is optionally multiply labeled. For example, the spectroscopically labeled protein can further comprise a second isotopically labeled amino acid comprising a second NMR active isotope. The second isotopically labeled amino acid can be a natural amino acid or an unnatural amino acid, and the labeling can be site-specific or uniform (e.g., the polypeptide backbone can be uniformly labeled with ¹⁵N, or the protein can be uniformly labeled with ¹³C, ²H, or ³H). Similarly, the isotopically labeled unnatural amino acid optionally includes more than one NMR active isotope, e.g., any combination of the isotopes listed herein.

In another class of embodiments, the unnatural amino acid comprises a fluorophore-labeled amino acid. In yet another class of embodiments, the unnatural amino acid comprises a spin-labeled amino acid, e.g., one comprising a nitroxide radical. In yet another class of embodiments, the unnatural amino acid comprises a chelator for a paramagnetic metal, e.g., an EDTA chelator for Mn²⁺, Cu²⁺, Zn²⁺, Co²⁺, or Gd³⁺. The paramagnetic metal is typically coordinated by the chelator.

In one class of embodiments, the translation system comprises (e.g., is in) a cell, for example, a prokaryotic cell (e.g., an E. coli cell) or a eukaryotic cell (e.g., a yeast or mammalian cell). The O-RS and/or O-tRNA are optionally encoded by one or more nucleic acids in the cell. The O-tRNA and the O-RS can be from the same organism (e.g., both from M. jannaschii or both from E. coli), or they can be from different organisms. As one example, the cell can comprise an E. coli cell, and the O-tRNA and the O-RS can comprise an M. jannaschii tyrosyl tRNA/tRNA synthetase pair. As another example, the cell can comprise a eukaryotic cell, and the O-tRNA and O-RS can comprise a prokaryotic orthogonal tRNA/tRNA synthetase pair. A variety of suitable orthogonal tRNA/tRNA synthetase pairs are known in the art. In other embodiments, the translation system comprises an in vitro translation system, e.g., a cellular extract.

In one aspect, the spectroscopically labeled protein is subjected to a spectroscopic technique, e.g., EPR spectroscopy, UV spectrometry, X-ray spectroscopy, mass spectroscopy, fluorescence spectroscopy, or vibrational (e.g., infrared or Raman) spectroscopy. In one preferred class of embodiments, the spectroscopic technique is NMR spectroscopy. A variety of single- and multi-dimensional NMR spectroscopic techniques have been described in the art and can be adapted for use in the methods, including, e.g., COSY, NOESY, HSQC, HSQC-NOESY, HETCOR, TROSY, SEA-TROSY, CRINEPT-TROSY, TROSY-HSQC, CRIPT-TROSY, PISEMA, MAS, and MAOSS. In one exemplary embodiment, the spectroscopically labeled protein comprises a ¹⁵N isotope, and the spectroscopic technique comprises a solvent-exposed amine transverse relaxation optimized spectroscopy (SEA-TROSY) experiment. In another exemplary embodiment, the spectroscopically labeled protein comprises a spin-label or a chelator coordinating a paramagnetic metal.

The spectroscopic technique is optionally performed on the spectroscopically labeled protein in vivo. Alternatively, the spectroscopic technique can be performed on the spectroscopically labeled protein in vitro, e.g., in a cellular extract, on a purified or partially purified protein, or the like.

The spectroscopic technique can be used, e.g., to obtain information about the structure, function, abundance, and/or dynamics of the protein. For example, in one class of embodiments, the methods include subjecting the spectroscopically labeled protein to a spectroscopic technique and generating information regarding one or more changes in structure or dynamics of the spectroscopically labeled protein.

The methods can be used to analyze ligand binding by the protein, conformational changes in the protein, catalytic mechanism, protein-protein interactions, and/or the like. Thus, in certain embodiments, the methods include analyzing an interaction between the spectroscopically labeled protein and a ligand or substrate. The interaction can include, e.g., a change in conformation in the spectroscopically labeled protein and/or a catalytic reaction performed by the spectroscopically labeled protein.

A second general class of embodiments provides methods for assigning NMR resonances to one or more amino acid residues in a protein of interest. In the methods, an unnatural amino acid comprising an NMR active isotope is provided and incorporated, producing an isotopically-labeled protein of interest, in a translation system. The translation system includes a nucleic acid encoding the protein of interest and comprising at least one selector codon for incorporating the unnatural amino acid at a specific site in the protein, an orthogonal tRNA (O-tRNA) that recognizes the selector codon, and an orthogonal aminoacyl tRNA synthetase (O-RS) that preferentially aminoacylates the O-tRNA with the unnatural amino acid. An NMR experiment is performed on the isotopically labeled protein, and data generated due to an interaction between the NMR active isotope of the unnatural amino acid and a proximal atom is analyzed, resulting in the assignment of one or more NMR resonances to one or more amino acid residues in the protein.

In one class of embodiments, the NMR active isotope is selected from the group consisting of: ⁷Li ¹³B ¹⁴N, ¹⁵N, ¹⁷O, ¹⁹F, ²³Na, ²⁷Al, ²⁹Si, ³¹P, ⁵⁹Co, ⁷⁷Se, ¹¹³Cd, ¹¹⁹Sn, ¹⁹⁵Pt, and a combination thereof.

Essentially all of the features noted above apply to this embodiment as well, as relevant, e.g., for NMR active isotopes, composition of the translation system, NMR techniques, and the like. For example, the NMR active isotope can comprise 15N, ²H, ¹⁹F, or ¹³C, among other examples. Similarly, the NMR experiment can be, e.g., a NOESY experiment, an HSQC experiment, an HSQC-NOESY experiment, a TROSY experiment, a SEA-TROSY experiment, or a TROSY-HSQC experiment.

The methods can be used to study protein structure and/or dynamics, e.g., two-dimensional structure, three-dimensional structure, ligand binding, catalysis, protein folding, and/or the like, e.g., even in large proteins difficult to analyze by other techniques. The site of incorporation of the unnatural amino acid can be chosen, for example, based on the particular aspect of the protein's structure and/or function that is of interest. Thus, for example, in one class of embodiments, the specific site of the unnatural amino acid comprises an active site or ligand binding site of the protein. In a related class of embodiments, the specific site of the unnatural amino acid comprises a site proximal to an active site or ligand binding site of the protein.

In one class of embodiments, the translation system comprises a cell. Data can be collected in vivo on the isotopically labeled protein, or it can be collected in vitro, e.g., on a cellular extract comprising the isotopically labeled protein, on a purified or partially purified isotopically labeled protein, or the like. In other embodiments, the translation system comprises an in vitro translation system, e.g., a cellular extract.

A related general class of embodiments provides methods for assigning an NMR resonance to an amino acid residue occupying a specific position in a protein of interest. The methods include providing a first sample comprising the protein, in which the protein comprises, at the specific position, an amino acid residue comprising an NMR active isotope. An NMR experiment is performed on the first sample and a first set of data is collected. A second sample comprising the protein is also provided, in which the protein comprises, at the specific position, an unnatural amino acid lacking the NMR active isotope. An NMR experiment is performed on the second sample and a second set of data is collected. The first and second sets of data are compared, whereby a resonance present in the first set and not present in the second set is assigned to the amino acid residue at the specific position.

In a preferred class of embodiments, the second sample is provided by translating a nucleic acid that encodes the protein in a translation system. The nucleic acid comprises a selector codon for incorporating the unnatural amino acid at the specific position in the protein. The translation system includes an orthogonal tRNA (O-tRNA) that recognizes the selector codon, the unnatural amino acid lacking the NMR active label, and an orthogonal aminoacyl tRNA synthetase (O-RS) that preferentially aminoacylates the O-tRNA with the unnatural amino acid. The NMR active isotope can be, e.g., ¹H, ¹⁵N, ¹³C, or ¹⁹F.

Essentially all of the features noted above apply to this embodiment as well, as relevant, e.g., for NMR active isotopes, composition of the translation system, NMR techniques, and the like.

Another general class of embodiments provides methods for producing and/or analyzing a spectroscopically labeled protein, where the spectroscopic label is attached to an unnatural amino acid after the unnatural amino acid is incorporated into the protein. In the methods, a nucleic acid that encodes the protein is translated in a translation system. The nucleic acid includes a selector codon for incorporating an unnatural amino acid at a specific position in the protein. The translation system includes an orthogonal tRNA (O-tRNA) that recognizes the selector codon, the unnatural amino acid, and an orthogonal aminoacyl tRNA synthetase (O-RS) that preferentially aminoacylates the O-tRNA with the unnatural amino acid. The unnatural amino acid is incorporated into the protein as it is translated, thereby producing a translated protein comprising the unnatural amino acid at the specific position. A spectroscopic label is attached (e.g., covalently attached) to the unnatural amino acid in the translated protein, thereby producing the spectroscopically labeled protein. The translated protein is optionally purified prior to attachment of the spectroscopic label.

In one class of embodiments, the spectroscopically labeled protein is subjected to a spectroscopic technique, which spectroscopic technique is NMR spectroscopy.

The unnatural amino acid can be essentially any unnatural amino acid to which a spectroscopic label can be attached. Suitable chemically reactive unnatural amino acids include, but are not limited to, p-acetyl-L-phenylalanine, m-acetyl-L-phenylalanine, O-allyl-L-tyrosine, O-(2-propynyl)-L-tyrosine, p-ethylthiocarbonyl-L-phenylalanine, p-(3-oxobutanoyl)-L-phenylalanine, p-azido-L-phenylalanine, and p-benzoyl-L-phenylalanine.

Similarly, the spectroscopic label can be essentially any spectroscopic label. For example, in one class of embodiments, the spectroscopic label comprises a fluorophore. As another example, the spectroscopic label can comprise an isotopic label, e.g., an NMR active isotope such as those described herein.

In one aspect, the spectroscopic label comprises a spin-label. For example, in one class of embodiments, the spin-label includes a nitroxide radical; e.g., the spin-label can be 2,2,6,6-tetramethyl-piperidine-1-oxyl (TEMPO) or 2,2,5,5-tetramethylpyrroline-1-oxyl. In a related class of embodiments, the spectroscopic label comprises a chelator for a paramagnetic metal, e.g., an EDTA chelator for Mn²⁺, Cu²⁺, Zn²⁺, Co²⁺, or Gd³⁺. In this class of embodiments, attaching the spectroscopic label to the unnatural amino acid optionally involves covalently attaching the chelator to the unnatural amino acid and associating the paramagnetic metal with the chelator. The metal can be associated with the chelator before or after attachment of the chelator to the unnatural amino acid.

In one aspect, the spectroscopically labeled protein is subjected to a spectroscopic technique, e.g., EPR spectroscopy, UV spectrometry, X-ray spectroscopy, mass spectroscopy, fluorescence spectroscopy, or vibrational (e.g., infrared or Raman) spectroscopy. In one preferred class of embodiments, the spectroscopic technique is NMR spectroscopy. In an exemplary class of NMR embodiments, the spectroscopic label comprises a chelator and a paramagnetic metal associated with the chelator. In another exemplary class of NMR embodiments, the spectroscopic label comprises a spin-label. In this class of embodiments, optionally an NMR experiment is performed on the spectroscopically labeled protein and a first set of data is collected, and then the spectroscopically labeled protein is reduced to provide a reduced form of the spectroscopically labeled protein, an NMR experiment is performed on the reduced form of the spectroscopically labeled protein, and a second set of data is collected.

The spectroscopic technique can be used, e.g., to obtain information about the structure, function, abundance, and/or dynamics of the protein. For example, in one class of embodiments, the methods include subjecting the spectroscopically labeled protein to a spectroscopic technique and generating information regarding a three-dimensional structure of the spectroscopically labeled protein. In one class of embodiments, the methods include subjecting the spectroscopically labeled protein to a spectroscopic technique and generating information regarding one or more changes in structure or dynamics of the spectroscopically labeled protein.

The methods can be used to analyze ligand binding by the protein, conformational changes in the protein, catalytic mechanism, protein-protein interactions, and/or the like. Thus, in certain embodiments, the methods include analyzing an interaction between the spectroscopically labeled protein and a ligand or substrate. The interaction can include, e.g., a change in conformation in the spectroscopically labeled protein and/or a catalytic reaction performed by the spectroscopically labeled protein.

Essentially all of the features noted above apply to this embodiment as well, as relevant, e.g., for composition of the translation system, NMR active isotopes, spectroscopic techniques, and the like.

Site-specific spectroscopically labeled proteins prepared by any of the methods herein form another feature of the invention. Similarly, systems comprising such a spectroscopically labeled protein and, e.g., a spectrometer are a feature of the invention.

### Definitions

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes combinations of two or more cells; reference to “a polynucleotide” includes, as a practical matter, many copies of that polynucleotide.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

Orthogonal: As used herein, the term “orthogonal” refers to a molecule (e.g., an orthogonal tRNA (O-tRNA) and/or an orthogonal aminoacyl tRNA synthetase (O-RS)) that functions with endogenous components of a cell or other translation system with reduced efficiency as compared to a corresponding molecule that is endogenous to the cell or translation system, or that fails to function when paired with endogenous components of the cell or translation system. In the context of tRNAs and aminoacyl-tRNA synthetases, orthogonal refers to an inability or reduced efficiency (e.g., less than 20% efficiency, less than 10% efficiency, less than 5% efficiency, or less than 1% efficiency), of an orthogonal tRNA to function with an endogenous tRNA synthetase compared to the ability of an appropriate (e.g., homologous or analogous) endogenous tRNA to function when paired with the endogenous complementary tRNA synthetase; or of an orthogonal aminoacyl-tRNA synthetase to function with an endogenous tRNA as compared to the ability of an appropriate endogenous tRNA synthetase to function when paired with the endogenous complementary tRNA. The orthogonal molecule lacks a functionally normal naturally occurring endogenous complementary molecule in the cell or translation system. For example, an orthogonal tRNA in a cell is aminoacylated by any endogenous RS of the cell with reduced or even undetectable efficiency, when compared to aminoacylation of an endogenous tRNA by the endogenous RS. In another example, an orthogonal RS aminoacylates any endogenous tRNA in a cell of interest with reduced or even undetectable efficiency, as compared to aminoacylation of the endogenous tRNA by a complementary endogenous RS. A second orthogonal molecule can be introduced into the cell that functions when paired with the first orthogonal molecule. For example, an orthogonal tRNA/RS pair includes introduced complementary components that function together in the cell with an efficiency (e.g., 45% efficiency, 50% efficiency, 60% efficiency, 70% efficiency, 75% efficiency, 80% efficiency, 90% efficiency, 95% efficiency, or 99% or more efficiency) as compared to that of a control, e.g., a corresponding (e.g., analogous) tRNA/RS endogenous pair, or an active orthogonal pair (e.g., a tyrosyl or tryptophanyl orthogonal tRNA/RS pair).

Orthogonal tRNA: As used herein, an orthogonal tRNA (O-tRNA) is a tRNA that is orthogonal to a translation system of interest. The O-tRNA can exist charged with an amino acid, or in an uncharged state. It will be appreciated that an O-tRNA of the invention is advantageously used to insert essentially any amino acid, whether natural or unnatural, into a growing polypeptide, during translation, in response to a selector codon.

Orthogonal amino acid synthetase: As used herein, an orthogonal amino acid synthetase (O-RS) is an enzyme that preferentially aminoacylates an O-tRNA with an amino acid in a translation system of interest.

Orthogonal tyrosyl-tRNA: As used herein, an orthogonal tyrosyl-tRNA (tyrosyl-O-tRNA) is a tRNA that is orthogonal to a translation system of interest, where the tRNA is: (1) identical or substantially similar to a naturally occurring tyrosyl-tRNA, (2) derived from a naturally occurring tyrosyl-tRNA by natural or artificial mutagenesis, (3) derived by any process that takes a sequence of a wild-type or mutant tyrosyl-tRNA sequence of (1) or (2) into account, or (4) homologous to a wild-type or mutant tyrosyl-tRNA. Exemplary tyrosyl-tRNAs are described in, e.g., Wang et al. (2001) Science 292:498 and U.S. patent application Ser. Nos. 10/126,927, 10/126,931, 10/825,867, and 60/634,151. The tyrosyl-tRNA can exist charged with an amino acid, or in an uncharged state. It is also to be understood that a “tyrosyl-O-tRNA” optionally is charged (aminoacylated) by a cognate synthetase with an amino acid other than tyrosine, e.g., with an unnatural amino acid. Indeed, it will be appreciated that a tyrosyl-O-tRNA of the invention is advantageously used to insert essentially any amino acid, whether natural or artificial, into a growing polypeptide, during translation, in response to a selector codon.

Orthogonal tyrosyl amino acid synthetase: As used herein, an orthogonal tyrosyl amino acid synthetase (tyrosyl-O-RS) is an enzyme that preferentially aminoacylates the tyrosyl-O-tRNA with an amino acid in a translation system of interest. The amino acid that the tyrosyl-O-RS loads onto the tyrosyl-O-tRNA can be any amino acid, whether natural, unnatural or artificial, and is not limited herein. The synthetase is optionally (1) the same as or homologous to a naturally occurring tyrosyl amino acid synthetase, (2) derived from a naturally occurring tyrosyl amino acid synthetase by natural or artificial mutagenesis, (3) derived by any process that takes a sequence of a wild-type or mutant tyrosyl amino acid synthetase sequence of (1) or (2) into account, or (4) homologous to a wild-type or mutant tyrosyl amino acid synthetase. Exemplary tyrosyl amino acid synthetases are described in, e.g., Wang et al. (2001) Science 292:498 and U.S. patent application Ser. Nos. 10/126,927, 10/126,931, 10/825,867, and 60/634,151.

Cognate: The term “cognate” refers to components that function together, e.g., an orthogonal tRNA and an orthogonal aminoacyl-tRNA synthetase that preferentially aminoacylates the orthogonal tRNA. The components can also be referred to as being complementary.

Preferentially aminoacylates: An O-RS “preferentially aminoacylates” a cognate O-tRNA when the O-RS charges the O-tRNA with an amino acid more efficiently than it charges any endogenous tRNA in an expression system. That is, when the O-tRNA and any given endogenous tRNA are present in a translation system in approximately equal molar ratios, the O-RS will charge the O-tRNA more frequently than it will charge the endogenous tRNA. Preferably, the relative ratio of O-tRNA charged by the O-RS to endogenous tRNA charged by the O-RS is high, preferably resulting in the O-RS charging the O-tRNA exclusively, or nearly exclusively, when the O-tRNA and endogenous tRNA are present in equal molar concentrations in the translation system. The relative ratio between O-tRNA and endogenous tRNA that is charged by the O-RS, when the O-tRNA and O-RS are present at equal molar concentrations, is greater than 1:1, preferably at least about 2:1, more preferably 5:1, still more preferably 10:1, yet more preferably 20:1, still more preferably 50:1, yet more preferably 75:1, and still more preferably 95:1, 98:1, 99:1, 100:1, 500:1, 1,000:1, 5,000:1 or higher.

The O-RS “preferentially aminoacylates an O-tRNA with an unnatural amino acid” when (a) the O-RS preferentially aminoacylates the O-tRNA compared to an endogenous tRNA, and (b) where that aminoacylation is specific for the unnatural amino acid, as compared to aminoacylation of the O-tRNA by the O-RS with any natural amino acid. That is, when the unnatural and natural amino acids are present in equal molar amounts in a translation system comprising the O-RS and O-tRNA, the O-RS will load the O-tRNA with the unnatural amino acid more frequently than with the natural amino acid. Preferably, the relative ratio of O-tRNA charged with the unnatural amino acid to O-tRNA charged with the natural amino acid is high. More preferably, O-RS charges the O-tRNA exclusively, or nearly exclusively, with the unnatural amino acid. The relative ratio between charging of the O-tRNA with the unnatural amino acid and charging of the O-tRNA with the natural amino acid, when both the natural and unnatural amino acids are present in the translation system in equal molar concentrations, is greater than 1:1, preferably at least about 2:1, more preferably 5:1, still more preferably 10:1, yet more preferably 20:1, still more preferably 50:1, yet more preferably 75:1, and still more preferably 95:1, 98:1, 99:1, 100:1, 500:1, 1,000:1, 5,000:1 or higher.

Selector codon: The term “selector codon” refers to a codon recognized by the O-tRNA in the translation process and not typically recognized by an endogenous tRNA. The O-tRNA anticodon loop recognizes the selector codon on the mRNA and incorporates its amino acid, e.g., an unnatural amino acid, such as a spectroscopically labeled amino acid, at this site in the polypeptide. Selector codons can include, e.g., nonsense codons, such as stop codons (e.g., amber, ochre, and opal codons), four or more base codons, rare codons, codons derived from natural or unnatural base pairs, and/or the like.

Translation system: The term “translation system” refers to the components that incorporate an amino acid into a growing polypeptide chain (protein). Components of a translation system can include, e.g., ribosomes, tRNAs, synthetases, mRNA and the like. The O-tRNA and/or the O-RSs of the invention can be added to or be part of an in vitro or in vivo translation system, e.g., in a non-eukaryotic cell, e.g., a bacterium (such as E. coli), or in a eukaryotic cell, e.g., a yeast cell, a mammalian cell, a plant cell, an algae cell, a ftungus cell, an insect cell, and/or the like.

Unnatural amino acid: As used herein, the term “unnatural amino acid” refers to any amino acid, modified amino acid, and/or amino acid analog, such as a spectroscopically labeled amino acid, that is not one of the 20 common naturally occurring amino acids or the rare natural amino acids selenocysteine or pyrrolysine.

Derived from: As used herein, the term “derived from” refers to a component that is isolated from or made using a specified molecule or organism, or information from the specified molecule or organism. For example, a polypeptide that is derived from a second polypeptide comprises an amino acid sequence that is identical or substantially similar to the amino acid sequence of the second polypeptide. In the case of polypeptides, the derived species can be obtained by, for example, naturally occurring mutagenesis, artificial directed mutagenesis or artificial random mutagenesis. The mutagenesis used to derive polypeptides can be intentionally directed or intentionally random. The mutagenesis of a polypeptide to create a different polypeptide derived from the first can be a random event (e.g., caused by polymerase infidelity) and the identification of the derived polypeptide can be serendipitous. Mutagenesis of a polypeptide typically entails manipulation of the polynucleotide that encodes the polypeptide.

Eukaryote: As used herein, the term “eukaryote” refers to organisms belonging to the Kingdom Eukarya. Eukaryotes are generally distinguishable from prokaryotes by their typically multicellular organization (but not exclusively multicellular; for example, yeast), the presence of a membrane-bound nucleus and other membrane-bound organelles, linear genetic material (i.e., linear chromosomes), the absence of operons, the presence of introns, message capping and poly-A mRNA, and other biochemical characteristics, such as a distinguishing ribosomal structure. Eukaryotic organisms include, for example, animals (e.g., mammals, insects, reptiles, birds, etc.), ciliates, plants (e.g., monocots, dicots, algae, etc.), fungi, yeasts, flagellates, microsporidia, protists, etc.

Prokaryote: As used herein, the term “prokaryote” refers to organisms belonging to the Kingdom Monera (also termed Prokarya). Prokaryotic organisms are generally distinguishable from eukaryotes by their unicellular organization, asexual reproduction by budding or fission, the lack of a membrane-bound nucleus or other membrane-bound organelles, a circular chromosome, the presence of operons, the absence of introns, message capping and poly-A mRNA, and other biochemical characteristics, such as a distinguishing ribosomal structure. The Prokarya include subkingdoms Eubacteria and Archaea (sometimes termed “Archaebacteria”). Cyanobacteria (the blue green algae) and mycoplasma are sometimes given separate classifications under the Kingdom Monera.

In response to: As used herein, the term “in response to” refers to the process in which a O-tRNA of the invention recognizes a selector codon and mediates the incorporation of the unnatural amino acid (e.g., the spectroscopically labeled unnatural amino acid), which is coupled to the tRNA, into the growing polypeptide chain.

Encode: As used herein, the term “encode” refers to any process whereby the information in a polymeric macromolecule or sequence string is used to direct the production of a second molecule or sequence string that is different from the first molecule or sequence string. As used herein, the term is used broadly, and can have a variety of applications. In one aspect, the term “encode” describes the process of semi-conservative DNA replication, where one strand of a double-stranded DNA molecule is used as a template to encode a newly synthesized complementary sister strand by a DNA-dependent DNA polymerase.

In another aspect, the term “encode” refers to any process whereby the information in one molecule is used to direct the production of a second molecule that has a different chemical nature from the first molecule. For example, a DNA molecule can encode an RNA molecule (e.g., by the process of transcription incorporating a DNA-dependent RNA polymerase enzyme). Also, an RNA molecule can encode a polypeptide, as in the process of translation. When used to describe the process of translation, the term “encode” also extends to the triplet codon that encodes an amino acid. In some aspects, an RNA molecule can encode a DNA molecule, e.g., by the process of reverse transcription incorporating an RNA-dependent DNA polymerase. In another aspect, a DNA molecule can encode a polypeptide, where it is understood that “encode” as used in that case incorporates both the processes of transcription and translation.

Nucleic acid: The term “nucleic acid” or “polynucleotide” encompasses any physical string of monomer units that can be corresponded to a string of nucleotides, including a polymer of nucleotides (e.g., a typical DNA or RNA polymer), PNAs, modified oligonucleotides (e.g., oligonucleotides comprising nucleotides that are not typical to biological RNA or DNA, such as 2′-O-methylated oligonucleotides), and the like. A nucleic acid can be e.g., single-stranded or double-stranded. Unless otherwise indicated, a particular nucleic acid sequence of this invention optionally comprises or encodes complementary sequences, in addition to any sequence explicitly indicated.

Polypeptide: A “polypeptide” (or a “protein”) is a polymer comprising two or more amino acid residues. The polymer can additionally comprise non-amino acid elements such as labels, quenchers, blocking groups, or the like and can optionally comprise modifications such as glycosylation or the like. The amino acid residues of the polypeptide can be natural and/or unnatural and can be unsubstituted, unmodified, substituted or modified.

Spectroscopic label: A “spectroscopic label” is a moiety (e.g., an atom or a chemical group) whose presence in a protein can produce a measurable difference in a spectroscopic property of the protein, as compared to the corresponding protein lacking the spectroscopic label. For example, in an unnatural amino acid comprising a spectroscopic label, one or more atoms of the unnatural amino acid can be replaced by or substituted with the spectroscopic label (e.g., an atom can be replaced by an isotopic label or be substituted with a spin-label), or the spectroscopic label can be added to the unnatural amino acid (e.g., a fluorophore or a nitroxide radical spin-label can be covalently attached to the unnatural amino acid). A “spectroscopically labeled protein” comprising an unnatural amino acid with a spectroscopic label (e.g., attached either before or after incorporation of the unnatural amino acid into the protein) thus displays a measurable difference in at least one spectroscopic property as compared to the protein including the unnatural amino acid but lacking the spectroscopic label.

Isotopically labeled: In an unnatural amino acid that is “isotopically labeled”, at least one atomic position in the amino acid is occupied exclusively or nearly exclusively by a single isotope of a given element, instead of being occupied by a mixture of the isotopes of that element at their natural abundance. The isotopic label can be the naturally most abundant isotope, or it can be a naturally less abundant isotope. Isotopic labels include, but are not limited to, NMR active isotopes and radioactive isotopes.

NMR active isotope: An “NMR active isotope” has a nonzero nuclear spin (e.g., a spin of ½).

Spin-label: A “spin-label” is a paramagnetic moiety. Spin-labels typically comprise unpaired electrons.

A variety of additional terms are defined or otherwise characterized herein.

### BRIEF DESCRIPTION OF THE DRAWINGS

FIG. 1 schematically illustrates a synthesis of ¹⁵N-labeled p-methoxyphenylalanine (2).

FIG. 2 shows a Gelcode Blue stained SDS-PAGE gel of purified ¹⁵N-MeOPhe-myoglobin. Lane 1 contains protein expressed in minimal media in the presence of 1 mM ¹⁵N-labeledp-methoxyphenylalanine (2); Lane 2 contains a sample expressed in the absence of ¹⁵N-labeledp-methoxyphenylalanine (2).

FIG. 3 presents a ¹H-¹⁵N HSQC NMR spectrum of ¹⁵N-MeOH-Phe4-labeled myoglobin (left) and non-labeled myoglobin (right). Cross sections along the nitrogen chemical shift of 120.6 ppm are shown above the 2D contour plots (¹H chemical shift, horizontal axis; ¹⁵N chemical shift, vertical axis).

### DETAILED DESCRIPTION

Although, with few exceptions, the genetic codes of all known organisms encode the same twenty amino acids, all that is required to add a new amino acid to the repertoire of an organism is a unique tRNA/aminoacyl-tRNA synthetase pair, a source of the amino acid, and a unique selector codon that specifies the amino acid (Furter (1998) Protein Sci., 7:419-426). The amber nonsense codon, TAG, together with orthogonal M. jannaschii and E. coli tRNA/synthetase pairs can be used to genetically encode a variety of amino acids with novel properties in E. coli (Wang et al., (2000) J. Am. Chem. Soc., 122:5010-5011; Wang et al., (2001) Science, 292:498-500; Wang et al., (2003) Proc. Natl. Acad. Sci. U.S.A., 100:56-61; Chin et al., (2002) Proc. Natl. Acad. Sci. U.S.A., 99:11020-11024; Wang and Schultz (2002) Chem. Commun. 1:1), and yeast (Chin and Schultz, (2002) ChemBioChem, 3:1135-1137; Chin et al. (2003) Science 301:964-967), respectively.

In order to add additional synthetic amino acids, such as spectroscopically labeled unnatural amino acids, to the genetic code, e.g., in vivo, orthogonal pairs of an aminoacyl-tRNA synthetase and a suitable tRNA are needed that can function efficiently in the translational machinery, but that are “orthogonal” to the translation system at issue, meaning that the pairs function independently of the synthetases and tRNAs endogenous to the translation system. Desired characteristics of an orthogonal pair include a tRNA that decodes or recognizes only a specific new codon, e.g., a selector codon, that is not decoded by any endogenous tRNA, and an aminoacyl-tRNA synthetase that preferentially aminoacylates (or charges) its cognate tRNA with only a specific non-natural amino acid. The O-tRNA is also desirably not aminoacylated by endogenous synthetases. For example, in E. coli, an orthogonal pair will include an aminoacyl-tRNA synthetase that does not cross-react with any of the endogenous tRNAs, e.g., of which there are 40 in E. coli, and an orthogonal tRNA that is not substantially aminoacylated by any of the endogenous synthetases, e.g., of which there are 21 in E. coli.

A number of such O-tRNA/O-RS pairs have been described, and others can be produced by one of skill in the art. Such O-tRNA/O-RS pairs can be used to incorporate a variety of different unnatural amino acids at specific sites in proteins of interest.

As noted, assignment of resonances to particular amino acids in protein NMR studies can be facilitated by site-specific labeling of one or more amino acids in the protein with an NMR active isotope. Site-specific, efficient incorporation of isotopically labeled unnatural amino acids into proteins can thus facilitate resonance assignment during NMR studies of proteins. For example, it can often be useful, e.g., in solution studies of protein-ligand interactions, protein conformational changes, or catalysis, to only assign the single residue(s) of an active site or a ligand binding site, using for example the SEA-TROSY experiment (Pellecchia et al. (2001) J. Am. Chem. Soc. 123:4633). Introducing one or several site-specific NMR labels at such locations can greatly simplify the assignment problem and can thus enable detailed NMR solution studies of even very large proteins. Similarly, site-specific introduction of one or more spin-labels or paramagnetic metals can facilitate NMR signal assignments.

Site-specific spectroscopic labeling of proteins can also be advantageous for use of spectroscopic techniques other than NMR (e.g., EPR spectroscopy, X-ray spectroscopy, mass spectroscopy, fluorescence spectroscopy, or vibrational (e.g., infrared or Raman) spectroscopy). For example, isotopic labeling can facilitate identification of peptide fragments in mass spectroscopy, incorporation of a fluorophore-containing unnatural amino acid (e.g., fluorophore-labeled L-phenylalanine or fluorophore-labeled p-acetyl-L-phenylalanine) can facilitate fluorescence spectroscopy, and incorporation of a spin-labeled unnatural amino acid can facilitate EPR.

Accordingly, one aspect of the invention provides methods for producing spectroscopically labeled proteins through site-specific incorporation of spectroscopically labeled unnatural amino acids into the proteins, using translation systems including orthogonal aminoacyl tRNA synthetases and orthogonal tRNAs. Another aspect provides methods for assigning NMR resonances by site-specifically incorporating isotopically labeled unnatural amino acids into proteins using such translation systems. Yet another aspect of the invention provides methods for producing spectroscopically labeled proteins through site-specific incorporation of unnatural amino acids into the proteins, using translation systems including orthogonal aminoacyl tRNA synthetases and orthogonal tRNAs, followed by attachment of spectroscopic labels to the unnatural amino acids.

Orthogonal tRNAS, Orthogonal Aminoacyl-tRNA Synthetases, and Pairs Thereof

Translation systems that are suitable for making proteins that include one or more unnatural amino acids are described, e.g., in International Publication Numbers WO 2002/086075, entitled “Methods and composition for the production of orthogonal tRNA-aminoacyl-tRNA synthetase pairs” and WO 2002/085923, entitled “In vivo incorporation of unnatural amino acids.” In addition, see International Application Number PCT/US2004/011786, filed Apr. 16, 2004, entitled “Expanding the Eukaryotic Genetic Code”. Each of these applications is incorporated herein by reference in its entirety. Such translation systems generally comprise cells (which can be non-eukaryotic cells such as E. coli or eukaryotic cells such as yeast) that include an orthogonal tRNA (O-tRNA), an orthogonal aminoacyl tRNA-synthetase (O-RS), and an unnatural amino acid (in the present invention, unnatural amino acids containing spectroscopic labels, e.g., isotopic labels, are examples of such unnatural amino acids), where the O-RS aminoacylates the O-tRNA with the unnatural amino acid.

In general, when an orthogonal pair (an O-tRNA, e.g., a suppressor tRNA, a frameshift tRNA, or the like, and an O-RS) recognizes a selector codon and loads an amino acid in response to the selector codon, the orthogonal pair is said to “suppress” the selector codon. That is, a selector codon that is not recognized by the translation system's (e.g., cell's) endogenous machinery is not ordinarily translated, which can result in blocking production of a polypeptide that would otherwise be translated from the nucleic acid. When an orthogonal pair is present, the O-RS aminoacylates the O-tRNA with an unnatural amino acid of interest, such as a spectroscopically labeled unnatural amino acid. The translation system (e.g., cell) uses the O-tRNA/O-RS pair to incorporate the unnatural amino acid into a growing polypeptide chain, e.g., via a nucleic acid that encodes a polypeptide (protein) of interest, where the nucleic acid comprises a selector codon that is recognized by the O-tRNA.

In certain embodiments of the invention, the translation system comprises a cell that includes an orthogonal aminoacyl-tRNA synthetase (O-RS), an orthogonal tRNA (O-tRNA), a spectroscopically labeled unnatural amino acid, and a nucleic acid that encodes a protein of interest, where the nucleic acid comprises the selector codon that is recognized by the O-tRNA. The cell can be a prokaryotic cell (such as an E. coli cell) or a eukaryotic cell (such as a yeast or mammalian cell). Typically, the orthogonal pair and the cell are derived from different sources (e.g., the cell can comprise an E. coli cell and the O-tRNA and the O-RS an M. jannaschii tyrosyl tRNA/tRNA synthetase pair, or the cell can comprise a eukaryotic cell and the O-tRNA and O-RS a prokaryotic orthogonal tRNA/tRNA synthetase pair). The translation system can also be a cell-free system, e.g., any of a variety of commercially available “in vitro” transcription/translation systems in combination with an O-tRNA/O-RS pair and an unnatural amino acid as described herein.

The cell or other translation system optionally includes multiple O-tRNA/O—RS pairs, which allows incorporation of more than one unnatural amino acid, e.g., two different spectroscopically labeled unnatural amino acids (comprising the same or different types of spectroscopic labels, e.g., isotopes) or a spectroscopically labeled unnatural amino acid and a different type of unnatural amino acid. For example, the cell can further include an additional different O-tRNA/O-RS pair and a second unnatural amino acid, where this additional O-tRNA recognizes a second selector codon and this additional O-RS preferentially aminoacylates the O-tRNA with the second unnatural amino acid. For example, a cell that includes an O-tRNA/O-RS pair (where the O-tRNA recognizes, e.g., an amber selector codon) can further comprise a second orthogonal pair, where the second O-tRNA recognizes a different selector codon (e.g., an opal codon, four-base codon, or the like). Desirably, the different orthogonal pairs are derived from different sources, which can facilitate recognition of different selector codons.

The O-tRNA and/or the O-RS can be naturally occurring or can be, e.g., derived by mutation of a naturally occurring tRNA and/or RS, e.g., by generating libraries of tRNAs and/or libraries of RSs, from any of a variety of organisms and/or by using any of a variety of available mutation strategies. For example, one strategy for producing an orthogonal tRNA/aminoacyl-tRNA synthetase pair involves importing a heterologous (to the host cell) tRNA/synthetase pair from, e.g., a source other than the host cell, or multiple sources, into the host cell. The properties of the heterologous synthetase candidate include, e.g., that it does not charge any host cell tRNA, and the properties of the heterologous tRNA candidate include, e.g., that it is not aminoacylated by any host cell synthetase. A second strategy for generating an orthogonal pair involves generating mutant libraries from which to screen and/or select an O-tRNA or O-RS. These strategies can also be combined.

Orthogonal tRNA (O-tRNA)

An orthogonal tRNA (O-tRNA) of the invention desirably mediates incorporation of an unnatural amino acid, such as a spectroscopically labeled unnatural amino acid, into a protein that is encoded by a nucleic acid that comprises a selector codon that is recognized by the O-tRNA, e.g., in vivo or in vitro. An O-tRNA can be provided to the translation system, e.g., a cell, as the O-tRNA or as a polynucleotide that encodes the O-tRNA or a portion thereof.

Methods of producing a recombinant orthogonal tRNA (O-tRNA) have been described and can be found, e.g., in international patent applications WO 2002/086075, entitled “Methods and compositions for the production of orthogonal tRNA-aminoacyl tRNA-synthetase pairs,” PCT/US2004/022187 entitled “Compositions of orthogonal lysyl-tRNA and aminoacyl-tRNA synthetase pairs and uses thereof,” and U.S. Ser. Nos. 60/479,931 and 60/496,548 entitled “Expanding the Eukaryotic Genetic Code.” See also Forster et al., (2003) “Programming peptidomimetic synthetases by translating genetic codes designed de novo” Proc. Natl. Acad. Sci. USA 100(11):6353-6357; and, Feng et al., (2003), “Expanding tRNA recognition of a tRNA synthetase by a single amino acid change” Proc. Natl. Acad. Sci. USA 100(10): 5676-5681, as well as other references herein.

Orthogonal Aminoacyl-tRNA Synthetase (O-RS)

An O-RS of the invention preferentially aminoacylates an O-tRNA with an unnatural amino acid such as a spectroscopically labeled unnatural amino acid, in vitro or in vivo. An O-RS of the invention can be provided to the translation system, e.g., a cell, by a polypeptide that includes an O-RS and/or by a polynucleotide that encodes an O-RS or a portion thereof.

Methods of producing O-RS, and altering the substrate specificity of the synthetase, have been described and can be found, e.g., in WO 2002/086075 entitled “Methods and compositions for the production of orthogonal tRNA-aminoacyl tRNA synthetase pairs,” and International Application Number PCT/US2004/011786, filed Apr. 16, 2004, and PCT/US2004/022187 entitled “Compositions of orthogonal lysyl-tRNA and aminoacyl-tRNA synthetase pairs and uses thereof”, filed Jul. 7, 2004, as well as other references herein.

O-tRNA/O-RS Pairs

A variety of O-tRNA/O-RS pairs capable of mediating the incorporation of unnatural amino acids into growing polypeptide chains has been described. For example, O-tRNA/O—RS pairs capable of mediating the incorporation of a variety of unnatural amino acids, including, e.g., O-methyl-L-tyrosine, L-3-(2-naphthyl)alanine, p-acetyl-L-phenylalanine, p-benzoyl-L-phenylalanine, p-azido-L-phenylalanine, and p-iodo-L-phenylalanine, are described in U.S. Ser. No. 10/126,927, U.S. Ser. Nos. 10/126,931, 10/825,867, and U.S. Ser. No. 60/602,048; O-tRNA/O-RS pairs capable of mediating the incorporation of keto amino acids are described in PCT/US 2003/32576; O-tRNA/O-RS pairs capable of mediating the incorporation of homoglutamine are described in PCT/US 2004/22187; 0-tRNA/O-RS pairs capable of mediating the incorporation of 5-hydroxytryptophan are described in U.S. Ser. No. 11/016,348; and O-tRNA/O-RS pairs capable of mediating the incorporation of alkynyl amino acids are described in U.S. Ser. No. 60/634,151.

Source and Host Organisms

The translational components of the invention can be derived from non-eukaryotic organisms. For example, the orthogonal O-tRNA can be derived from a non-eukaryotic organism (or a combination of organisms), e.g., an archaebacterium, such as Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, Methanococcus maripaludis, Methanopyrus kandleri, Methanosarcina mazei, Pyrobaculum aerophilum, Pyrococcus abyssi, Sulfolobus solfataricus, Sulfolobus tokodaii, Thermoplasma acidophilum, Thermoplasma volcanium, or the like, or a eubacterium, such as Escherichia coli, Thermus thermophilus, Bacillus stearothermphilus, or the like, while the orthogonal O—RS can be derived from a non-eukaryotic organism (or a combination of organisms), e.g., an archaebacterium, such as Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, Methanococcus maripaludis, Methanopyrus kandleri, Methanosarcina mazei, Pyrobaculum aerophilum, Pyrococcus abyssi, Sulfolobus solfataricus, Sulfolobus tokodaii, Thermoplasma acidophilum, Thermoplasma volcanium, or the like, or a eubacterium, such as Escherichia coli, Thermus thermophilus, Bacillus stearothermphilus, or the like. In one embodiment, eukaryotic sources, e.g., plants, algae, protists, fingi, yeasts, animals (e.g., mammals, insects, arthropods, etc.), or the like, can also be used as sources of O-tRNAs and O-RSs.

The individual components of an O-tRNA/O-RS pair can be derived from the same organism or different organisms. In one embodiment, the O-tRNA/O-RS pair is from the same organism. Alternatively, the O-tRNA and the O-RS of the O-tRNA/O-RS pair are from different organisms.

The O-tRNA, O-RS or O-tRNA/O-RS pair can be selected or screened in vivo or in vitro and/or used in a cell, e.g., a prokaryotic (non-eukaryotic) cell or a eukaryotic cell, to produce a polypeptide with an unnatural amino acid of interest. A non-eukaryotic cell can be from any of a variety of sources, e.g., a eubacterium, such as Escherichia coli, Thermus thermophilus, Bacillus stearothermphilus, or the like, or an archaebacterium, such as Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, Methanococcus maripaludis, Methanopyrus kandleri, Methanosarcina mazei, Pyrobaculum aerophilum, Pyrococcus abyssi, Sulfolobus solfataricus, Sulfolobus tokodaii, Thermoplasma acidophilum, Thermoplasma volcanium, or the like. A eukaryotic cell can be from any of a variety of sources, e.g., a plant (e.g., a complex plant such as a monocot or a dicot), an algae, a protist, a fungus, a yeast (e.g., Saccharomyces cerevisiae), an animal (e.g., a mammal, an insect, an arthropod, etc.), or the like. For example, suitable insect host cells include, but are not limited to, Lepidopteran, Spodoptera frugiperda, Bombyx mori, Heliothis virescens, Heliothis zea, Mamestra brassicas, Estigmene acrea, and Trichoplusia ni insect cells; exemplary insect cell lines include BT1-TN-5B1-4 (High Five), BTI-TN-MG1, Sf9, Sf21, TN-368, D.Mel-2, and Schneider S-2 cells, among many others. To express a protein incorporating an unnatural amino acid, such insect cells are optionally infected with a recombinant baculovirus vector encoding the protein and a selector codon. A variety of baculovirus expression systems are known in the art and/or are commercially available, e.g., BaculoDirect™ (Invitrogen, Carlsbad, Calif.) and BD BaculoGold™ Baculovirus Expression Vector System (BD Biosciences, San Jose, Calif.). Compositions of cells with translational components of the invention are also a feature of the invention.

See also, International Application Number PCT/US2004/011786, filed Apr. 16, 2004, for screening O-tRNA and/or O-RS in one species for use in another species.

Selector Codons

Selector codons of the invention expand the genetic codon framework of the protein biosynthetic machinery. For example, a selector codon includes, e.g., a unique three base codon, a nonsense codon, such as a stop codon, e.g., an amber codon (UAG), or an opal codon (UGA), an unnatural codon, at least a four base codon (e.g., AGGA), a rare codon, or the like. A number of selector codons can be introduced into a desired gene, e.g., one or more, two or more, more than three, etc. By using different selector codons, multiple orthogonal tRNA/synthetase pairs can be used that allow the simultaneous site-specific incorporation of multiple different unnatural amino acids into the protein of interest, using these different selector codons. Similarly, more than one copy of a given selector codon can by introduced into a desired gene to allow the site-specific incorporation of a given unnatural amino acid at multiple sites (e.g., two or more, three or more, etc.) in the protein of interest.

Conventional site-directed mutagenesis can be used to introduce the selector codon at the site of interest in a nucleic acid encoding a polypeptide of interest. When the O-RS, O-tRNA and the nucleic acid that encodes a polypeptide of interest are combined, e.g., in vivo, the spectroscopically labeled unnatural amino acid is incorporated in response to the selector codon to give a polypeptide containing the spectroscopically labeled unnatural amino acid at the specified position.

The incorporation of unnatural amino acids such as spectroscopically labeled unnatural amino acids in vivo can be done without significant perturbation of the host cell. For example, in non-eukaryotic cells, such as Escherichia coli, because the suppression efficiency of a stop selector codon, the UAG codon, depends upon the competition between the O-tRNA, e.g., the amber suppressor tRNA, and release factor 1 (RF1) (which binds to the UAG codon and initiates release of the growing peptide from the ribosome), the suppression efficiency can be modulated by, e.g., either increasing the expression level of O-tRNA, e.g., the suppressor tRNA, or using an RF1 deficient strain. In eukaryotic cells, because the suppression efficiency for a UAG codon depends upon the competition between the O-tRNA, e.g., the amber suppressor tRNA, and a eukaryotic release factor (e.g., eRF) (which binds to a stop codon and initiates release of the growing peptide from the ribosome), the suppression efficiency can be modulated by, e.g., increasing the expression level of O-tRNA, e.g., the suppressor tRNA. In addition, additional compounds can also be present that modulate release factor action, e.g., reducing agents such as dithiothreitol (DTT).

Unnatural amino acids, including, e.g., spectroscopically labeled unnatural amino acids, can also be encoded with rare codons. For example, when the arginine concentration in an in vitro protein synthesis reaction is reduced, the rare arginine codon, AGG, has proven to be efficient for insertion of Ala by a synthetic tRNA acylated with alanine. See, e.g., Ma et al., Biochemistry, 32:7939 (1993). In this case, the synthetic tRNA competes with the naturally occurring tRNA_{Arg}, which exists as a minor species in Escherichia coli. In addition, some organisms do not use all triplet codons. An unassigned codon AGA in Micrococcus luteus has been utilized for insertion of amino acids in an in vitro transcription/translation extract. See, e.g., Kowal and Oliver, Nucl. Acid. Res., 25:4685 (1997). Components of the invention can be generated to use these rare codons in vivo.

Selector codons can also comprise extended codons, e.g., four or more base codons, such as four, five, six or more base codons. Examples of four base codons include, e.g., AGGA, CUAG, UAGA, CCCU, and the like. Examples of five base codons include, e.g., AGGAC, CCCCU, CCCUC, CUAGA, CUACU, UAGGC, and the like. Methods of the invention can include using extended codons based on frameshift suppression. Four or more base codons can insert, e.g., one or multiple unnatural amino acids into the same protein. In other embodiments, the anticodon loops can decode, e.g., at least a four-base codon, at least a five-base codon, or at least a six-base codon or more. Since there are 256 possible four-base codons, multiple unnatural amino acids can be encoded in the same cell using a four or more base codon. See also, Anderson et al. (2002) “Exploring the Limits of Codon and Anticodon Size” Chemistry and Biology, 9:237-244; and, Magliery (2001) “Expanding the Genetic Code: Selection of Efficient Suppressors of Four-base Codons and Identification of ‘Shifty’ Four-base Codons with a Library Approach in Escherichia coli” J. Mol. Biol. 307: 755-769.

For example, four-base codons have been used to incorporate unnatural amino acids into proteins using in vitro biosynthetic methods. See, e.g., Ma et al., (1993) Biochemistry, 32:7939; and Hohsaka et al., (1999) J. Am. Chem. Soc., 121:34. CGGG and AGGU were used to simultaneously incorporate 2-naphthylalanine and an NBD derivative of lysine into streptavidin in vitro with two chemically acylated frameshift suppressor tRNAs. See, e.g., Hohsaka et al., (1999) J. Am. Chem. Soc., 121:12194. In an in vivo study, Moore et al. examined the ability of tRNA^{Leu} derivatives with NCUA anticodons to suppress UAGN codons (N can be U, A, G, or C), and found that the quadruplet UAGA can be decoded by a tRNA^{Leu} with a UCUA anticodon with an efficiency of 13 to 26% with little decoding in the 0 or −1 frame. See Moore et al., (2000) J. Mol. Biol., 298:195. In one embodiment, extended codons based on rare codons or nonsense codons can be used in the invention, which can reduce missense readthrough and frameshift suppression at other unwanted sites.

For a given system, a selector codon can also include one of the natural three base codons, where the endogenous system does not use (or rarely uses) the natural base codon. For example, this includes a system that is lacking a tRNA that recognizes the natural three base codon, and/or a system where the three base codon is a rare codon.

Selector codons optionally include unnatural base pairs. These unnatural base pairs further expand the existing genetic alphabet. One extra base pair increases the number of triplet codons from 64 to 125. Properties of third base pairs include stable and selective base pairing, efficient enzymatic incorporation into DNA with high fidelity by a polymerase, and the efficient continued primer extension after synthesis of the nascent unnatural base pair. Descriptions of unnatural base pairs which can be adapted for methods and compositions of the invention include, e.g., Hirao, et al., (2002) “An unnatural base pair for incorporating amino acid analogues into protein” Nature Biotechnology, 20:177-182. See also Wu, Y., et al., (2002) J. Am. Chem. Soc. 124:14626-14630. Other relevant publications are listed below.

For in vivo usage, the unnatural nucleoside is membrane permeable and is phosphorylated to form the corresponding triphosphate. In addition, the increased genetic information is stable and not destroyed by cellular enzymes. Previous efforts by Benner and others took advantage of hydrogen bonding patterns that are different from those in canonical Watson-Crick pairs, the most noteworthy example of which is the iso-C:iso-G pair. See, e.g., Switzer et al., (1989) J. Am. Chem. Soc., 111:8322; and Piccirilli et al., (1990) Nature, 343:33; Kool, (2000) Curr. Opin. Chem. Biol., 4:602. These bases in general mispair to some degree with natural bases and cannot be enzymatically replicated. Kool and co-workers demonstrated that hydrophobic packing interactions between bases can replace hydrogen bonding to drive the formation of base pair. See Kool, (2000) Curr. Opin. Chem. Biol 4:602; and Guckian and Kool, (1998) Angew. Chem. Int. Ed. Engl., 36, 2825. In an effort to develop an unnatural base pair satisfying all the above requirements, Schultz, Romesberg and co-workers have systematically synthesized and studied a series of unnatural hydrophobic bases. A PICS:PICS self-pair is found to be more stable than natural base pairs, and can be efficiently incorporated into DNA by Klenow fragment of Escherichia coli DNA polymerase I (KF). See, e.g., McMinn et al., (1999) J. Am. Chem. Soc., 121:11586; and Ogawa et al., (2000) J. Am. Chem. Soc., 122:3274. A 3MN:3MN self-pair can be synthesized by KF with efficiency and selectivity sufficient for biological function. See, e.g., Ogawa et al., (2000) J. Am. Chem. Soc., 122:8803. However, both bases act as a chain terminator for further replication. A mutant DNA polymerase has been recently evolved that can be used to replicate the PICS self pair. In addition, a 7AI self pair can be replicated. See, e.g., Tae et al., (2001) J. Am. Chem. Soc., 123:7439. A novel metallobase pair, Dipic:Py, has also been developed, which forms a stable pair upon binding Cu(II). See Meggers et al., (2000) J. Am. Chem. Soc., 122:10714. Because extended codons and unnatural codons are intrinsically orthogonal to natural codons, the methods of the invention can take advantage of this property to generate orthogonal tRNAs for them.

A translational bypassing system can also be used to incorporate a spectroscopically labeled unnatural amino acid or other unnatural amino acid into a desired polypeptide. In a translational bypassing system, a large sequence is inserted into a gene but is not translated into protein. The sequence contains a structure that serves as a cue to induce the ribosome to hop over the sequence and resume translation downstream of the insertion.

Unnatural Amino Acids

As used herein, an unnatural amino acid refers to any amino acid, modified amino acid, or amino acid analog other than selenocysteine and/or pyrrolysine and the following twenty genetically encoded alpha-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. The generic structure of an alpha-amino acid is illustrated by Formula I:

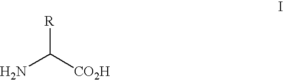

An unnatural amino acid is typically any structure having Formula I wherein the R group is any substituent other than one used in the twenty natural amino acids. See e.g., Biochemistry by L. Stryer, 3^{rd} ed. 1988, Freeman and Company, New York, for structures of the twenty natural amino acids. Note that the unnatural amino acids of the invention can be naturally occurring compounds other than the twenty alpha-amino acids above (or, of course, can be artificially produced synthetic compounds).

Because the unnatural amino acids of the invention typically differ from the natural amino acids in side chain, the unnatural amino acids form amide bonds with other amino acids, e.g., natural or unnatural, in the same manner in which they are formed in naturally occurring proteins. However, the unnatural amino acids have side chain groups that distinguish them from the natural amino acids.

In unnatural amino acids, for example, R in Formula I optionally comprises an alkyl-, aryl-, acyl-, keto-, azido-, hydroxyl-, hydrazine, cyano-, halo-, hydrazide, alkenyl, alkynyl, ether, thiol, seleno-, sulfonyl-, borate, boronate, phospho, phosphono, phosphine, heterocyclic, enone, imine, aldehyde, ester, thioacid, hydroxylamine, amine, or the like, or any combination thereof. Other unnatural amino acids of interest include, but are not limited to, amino acids comprising a photoactivatable cross-linker, spin-labeled amino acids, fluorescent amino acids, fluorophore-labeled amino acids, luminescent amino acids, metal binding amino acids, metal-containing amino acids, radioactive amino acids, amino acids with novel functional groups, amino acids that covalently or noncovalently interact with other molecules, photocaged and/or photoisomerizable amino acids, biotin or biotin-analog containing amino acids, keto containing amino acids, glycosylated amino acids, amino acids comprising polyethylene glycol or polyether, chemically cleavable or photocleavable amino acids, amino acids with an elongated side chain as compared to natural amino acids (e.g., polyethers or long chain hydrocarbons, e.g., greater than about 5, greater than about 10 carbons, etc.), carbon-linked sugar-containing amino acids, redox-active amino acids, amino thioacid containing amino acids, heavy atom-containing amino acids, spectroscopically labeled unnatural amino acids, and amino acids containing one or more toxic moiety. In some embodiments, the unnatural amino acids have a photoactivatable cross-linker. In one embodiment, the unnatural amino acids have a saccharide moiety attached to the amino acid side chain and/or other carbohydrate modification.

In addition to unnatural amino acids that contain novel side chains, unnatural amino acids also optionally comprise modified backbone structures, e.g., as illustrated by the structures of Formula II and III: 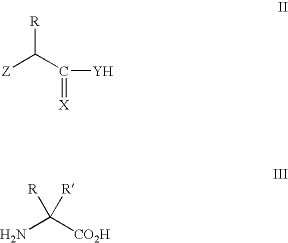
wherein Z typically comprises OH, NH2, SH, NH—R′, or S—R′; X and Y, which can be the same or different, typically comprise S or O, and R and R′, which are optionally the same or different, are typically selected from the same list of constituents for the R group described above for the unnatural amino acids having Formula I as well as hydrogen. For example, unnatural amino acids of the invention optionally comprise substitutions in the amino or carboxyl group as illustrated by Formulas II and III. Unnatural amino acids of this type include, but are not limited to, α-hydroxy acids, α-thioacids α-aminothiocarboxylates, e.g., with side chains corresponding to the common twenty natural amino acids or unnatural side chains. In addition, substitutions at the α-carbon optionally include L, D, or α-α-disubstituted amino acids such as D-glutamate, D-alanine, D-methyl-O-tyrosine, aminobutyric acid, and the like. Other structural alternatives include cyclic amino acids, such as proline analogs as well as 3,4,6,7,8, and 9 membered ring proline analogs, β and γ amino acids such as substituted β-alanine and γ-amino butyric acid. Additional unnatural amino acid structures of the invention include homo-beta-type structures, e.g., where there is, e.g., a methylene or amino group sandwiched adjacent to the alpha carbon, e.g., isomers of homo-beta-tyrosine, alpha-hydrazino-tyrosine. See, e.g.,

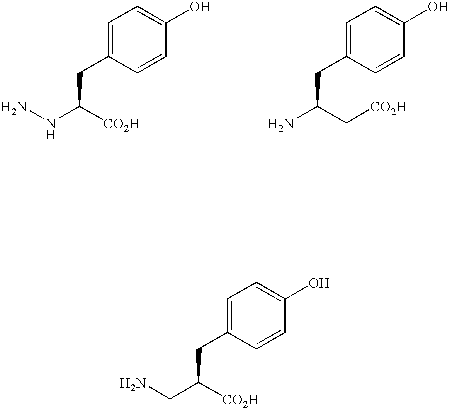

Many unnatural amino acids are based on natural amino acids, such as tyrosine, glutamine, phenylalanine, and the like. For example, tyrosine analogs include para-substituted tyrosines, ortho-substituted tyrosines, and meta substituted tyrosines, wherein the substituted tyrosine comprises an acetyl group, a benzoyl group, an amino group, a hydrazine, an hydroxyamine, a thiol group, a carboxy group, an isopropyl group, a methyl group, a C₆-C₂₀ straight chain or branched hydrocarbon, a saturated or unsaturated hydrocarbon, an O-methyl group, a polyether group, a nitro group, a halogen atom, or the like. In addition, multiply substituted aryl rings are also contemplated. Glutamine analogs of the invention include, but are not limited to, α-hydroxy derivatives, γ-substituted derivatives, cyclic derivatives, and amide substituted glutamine derivatives. Example phenylalanine analogs include, but are not limited to, para-substituted phenylalanines, ortho-substituted phenyalanines, and meta-substituted phenylalanines, wherein the substituent comprises a hydroxy group, a methoxy group, a methyl group, an allyl group, an aldehyde or keto group, a halogen atom, or the like. Specific examples of unnatural amino acids include, but are not limited to, homoglutamine, a 3,4-dihydroxy-L-phenylalanine, a p-acetyl-L-phenylalanine, an m-acetyl-L-phenylalanine, a p-propargyloxy-phenylalanine, an O-methyl-L-tyrosine (also known as p-methoxy-phenylalanine), an L-3-(2-naphthyl)alanine, a 3-methyl-phenylalanine, an O-4-allyl-L-tyrosine, a 4-propyl-L-tyrosine, an O-(2-propynyl)-L-tyrosine, a p-ethylthiocarbonyl-L-phenylalanine, a p-(3-oxobutanoyl)-L-phenylalanine, a tri-O-acetyl-β-GlcNAc-L-serine, a tri-O-acetyl-α-GalNAc-L-threonine, a β-GlcNAc-serine, an α-GalNAc-threonine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-amino-L-phenylalanine, an isopropyl-L-phenylalanine, a p-bromo-L-phenylalanine (also known as L-4-bromophenylalanine), an L-3-bromophenylalanine, an L-2-bromophenylalanine, an L-3-bromotyrosine, an L-2-bromotyrosine, and the like.

No attempt is made to identify all possible unnatural amino acids, any of which can be modified to include a spectroscopic label (e.g., if one is not already included; as noted above, certain unnatural amino acids, e.g., spin-labeled amino acids, fluorophore-labeled amino acids, and the like, can already include a spectroscopic label). A few examples of spectroscopically labeled unnatural amino acids follow, but it will be evident to one of skill that an extremely large number of labeled unnatural amino acids can be adapted for use in the present invention.

In one aspect, the spectroscopically labeled unnatural amino acid comprises an isotopically labeled unnatural amino acid. For example, the unnatural amino acid can include a radioactive isotope or an NMR active isotope. A variety of NMR active isotopes are known in the art, including, but not limited to, ²H, ¹³C, ¹⁵N, ³H, ⁷Li, ¹³B, ¹⁴N, ¹⁷O, ¹⁹F, ²³Na, ²⁷Al, ²⁹Si, ³¹P, ³⁵Cl, ³⁷Cl, ³⁹K, ⁵⁹Co, ⁷⁷Se, ⁸¹Br, ¹¹³Cd, ¹¹⁹Sn, and ¹⁹⁵Pt.

The NMR active (or other) isotope can be attached to or incorporated into the unnatural amino acid at essentially any convenient position (e.g., the isotope can be an addition to the unnatural amino acid, or it can replace an atom in the unnatural amino acid). As just a few examples, the NMR active isotope can be part of a methyl group, an amino group, an azido group, a keto group, a carboxy group, a cyano group, an alkyl group, an alkoxy group, an alkynyl moiety, a thiol group, a halogen atom, an aryl group, a sugar residue, a photocrosslinking moiety, or a photolabile group.

As one example, essentially any unnatural amino acid can be isotopically labeled by replacing the nitrogen of the alpha-amino group with ¹⁵N. For example, such labeling of p-methoxyphenylalanine produces ¹⁵N-labeled p-methoxyphenylalanine.

As another example, a methyl group on an unnatural amino acid such as O-methyl-L-tyrosine (also called p-methoxyphenylalanine) can be replaced by an isotopically (e.g., ¹³C, ²H, and/or ³H) labeled methyl group. Carbon and hydrogen isotopes can similarly be incorporated at a large number of positions in essentially any unnatural amino acid.

As yet another example, phosphorus-containing unnatural amino acids (e.g. L-phosphoserine, L-phosphotyrosine, L-phosphothreonine, phosphonoserine, or phosphonotyrosine) can be isotopically labeled with ³¹P. As yet another example, a brominated unnatural amino acid (e.g., p-bromo-L-phenylalanine, L-3-bromophenylalanine, L-2-bromophenylalanine, L-3-bromotyrosine, or L-2-bromotyrosine) can be isotopically labeled with ⁸¹Br. Similarly, unnatural amino acids can incorporate ¹⁹F, or essentially any other convenient isotopic label.

In another aspect, the spectroscopically labeled unnatural amino acid comprises a spin-labeled amino acid. Such labels can, e.g., be useful in NMR and/or EPR. In one class of embodiments, the spin-labeled amino acid comprises a nitroxide radical (e.g., 2,2,6,6-tetramethyl-piperidine-1-oxyl (TEMPO) or 2,2,5,5-tetramethylpyrroline-1-oxyl). An exemplary spin-labeled amino acid is 4-amino-2,2,6,6-tetramethyl piperidine-1-oxyl-4-carboxylic acid (TOAC); see also spin-labeled amino acids 1-3 of Cornish et al. (1994) “Site-specific incorporation of biophysical probes into proteins” Proc. Natl. Acad. Sci. USA 91:2910-4. Similarly, the unnatural amino acid can comprise a chelator for a paramagnetic metal, e.g., an EDTA chelator for a paramagnetic metal such as Mn²⁺, Cu²⁺, Zn²⁺, Co²⁺, or Gd³⁺. Exemplary paramagnetic metals include, but are not limited to, Mn²⁺, Cu²⁺, Zn²⁺, Co²⁺, Gd³⁺, Ce³⁺, Tb³⁺, Dy³⁺, Ho³⁺, Er³⁺, Tm³⁺, Yb³⁺, and other lanthanides. See, e.g., Pintacuda et al. (2004) J. Biomolec. NMR 29:351-361; Jahnke (2002) ChemBioChem 3:167-173; Jahnke et al. (2001) J. Am. Chem. Soc. 123:3149-3150; and Jahnke et al. (2000) J. Am. Chem. Soc. 122:7394-7395.

Chemical Synthesis of Unnatural Amino Acids

Many of the unnatural amino acids provided above are commercially available, e.g., from Sigma (USA) or Aldrich (Milwaukee, Wis., USA). Those spectroscopically labeled unnatural amino acids that are not commercially available are optionally synthesized as provided in various publications or using standard methods known to those of skill in the art. For organic synthesis techniques, see, e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York). Additional publications describing the synthesis of unnatural amino acids include, e.g., WO 2002/085923 entitled “In vivo incorporation of Unnatural Amino Acids;” Matsoukas et al. (1995) J. Med. Chem. 38:4660-4669; King and Kidd (1949) “A New Synthesis of Glutamine and of γ-Dipeptides of Glutamic Acid from Phthylated Intermediates” J. Chem. Soc. 3315-3319; Friedman and Chatterrji (1959) “Synthesis of Derivatives of Glutamine as Model Substrates for Anti-Tumor Agents” J. Am. Chem. Soc. 81:3750-3752; Craig et al. (1988) “Absolute Configuration of the Enantiomers of 7-Chloro-4 [[4-(diethylamino)-1-methylbutyl]amino]quinoline (Chloroquine)” J. Org. Chem. 53:1167-1170; Azoulay et al. (1991) “Glutamine analogues as Potential Antimalarials” Eur. J. Med. Chem. 26:201-205; Koskinen and Rapoport (1989) “Synthesis of 4-Substituted Prolines as Conformationally Constrained Amino Acid Analogues” J. Org. Chem. 54:1859-1866; Christie and Rapoport (1985) “Synthesis of Optically Pure Pipecolates from L-Asparagine: Application to the Total Synthesis of (+)-Apovincamine through Amino Acid Decarbonylation and Iminium Ion Cyclization” J. Org. Chem. 1989:1859-1866; Barton et al. (1987) “Synthesis of Novel α-Amino-Acids and Derivatives Using Radical Chemistry: Synthesis of L- and D-α-Amino-Adipic Acids, L-α-aminopimelic Acid and Appropriate Unsaturated Derivatives” Tetrahedron Lett. 43:4297-4308; and, Subasinghe et al. (1992) “Quisqualic acid analogues: synthesis of beta-heterocyclic 2-aminopropanoic acid derivatives and their activity at a novel quisqualate-sensitized site” J. Med. Chem. 35:4602-4607. See also International Application Number PCT/US03/41346, entitled “Protein Arrays,” filed on Dec. 22, 2003.

Cellular Uptake of Unnatural Amino Acids

Unnatural amino acid uptake by a cell is one issue that is typically considered when designing and selecting unnatural amino acids, e.g., for incorporation into a protein. For example, the high charge density of α-amino acids suggests that these compounds are unlikely to be cell permeable. Natural amino acids are taken up into the cell via a collection of protein-based transport systems often displaying varying degrees of amino acid specificity. A rapid screen can be done which assesses which unnatural amino acids, if any, are taken up by cells. See, e.g., toxicity assays in, e.g., International Application Number PCT/US03/41346, supra, and Liu and Schultz (1999) “Progress toward the evolution of an organism with an expanded genetic code” Proc. Natl. Acad. Sci. USA 96:4780-4785. Although uptake is easily analyzed with various assays, an alternative to designing unnatural amino acids that are amenable to cellular uptake pathways is to provide biosynthetic pathways to create amino acids in vivo.

Biosynthesis of Unnatural Amino Acids

Many biosynthetic pathways already exist in cells for the production of amino acids and other compounds. While a biosynthetic method for a particular unnatural amino acid may not exist in nature, e.g., in a cell, the invention provides such methods. For example, biosynthetic pathways for unnatural amino acids are optionally generated in host cell by adding new enzymes or modifying existing host cell pathways. Additional new enzymes are optionally naturally occurring enzymes or artificially evolved enzymes. For example, the biosynthesis of p-aminophenylalanine (as presented in an example in WO 2002/085923, supra) relies on the addition of a combination of known enzymes from other organisms. The genes for these enzymes can be introduced into a cell by transforming the cell with a plasmid comprising the genes. The genes, when expressed in the cell, provide an enzymatic pathway to synthesize the desired compound. Examples of the types of enzymes that are optionally added are provided in the examples below. Additional enzyme sequences are found, e.g., in Genbank. Artificially evolved enzymes are also optionally added into a cell in the same manner. In this manner, the cellular machinery and resources of a cell are manipulated to produce unnatural amino acids.

Indeed, any of a variety of methods can be used for producing novel enzymes for use in biosynthetic pathways, or for evolution of existing pathways, for the production of unnatural amino acids, in vitro or in vivo. Many available methods of evolving enzymes and other biosynthetic pathway components can be applied to the present invention to produce unnatural amino acids (or, indeed, to evolve synthetases to have new substrate specificities or other activities of interest). For example, DNA shuffling is optionally used to develop novel enzymes and/or pathways of such enzymes for the production of unnatural amino acids (or production of new synthetases), in vitro or in vivo. See, e.g., Stemmer (1994) “Rapid evolution of a protein in vitro by DNA shuffling” Nature 370(4):389-391; and Stemmer (1994) “DNA shuffling by random fragmentation and reassembly: In vitro recombination for molecular evolution” Proc. Natl. Acad. Sci. USA. 91:10747-10751. A related approach shuffles families of related (e.g., homologous) genes to quickly evolve enzymes with desired characteristics. An example of such “family gene shuffling” methods is found in Crameri et al. (1998) “DNA shuffling of a family of genes from diverse species accelerates directed evolution” Nature 391(6664):288-291. New enzymes (whether biosynthetic pathway components or synthetases) can also be generated using a DNA recombination procedure known as “incremental truncation for the creation of hybrid enzymes” (“ITCHY”), e.g., as described in Ostermeier et al. (1999) “A combinatorial approach to hybrid enzymes independent of DNA homology” Nature Biotech 17:1205. This approach can also be used to generate a library of enzyme or other pathway variants which can serve as substrates for one or more in vitro or in vivo recombination methods. See, also, Ostermeier et al. (1999) “Combinatorial Protein Engineering by Incremental Truncation” Proc. Natl. Acad. Sci. USA 96: 3562-67, and Ostermeier et al. (1999) “Incremental Truncation as a Strategy in the Engineering of Novel Biocatalysts” Biological and Medicinal Chemistry 7: 2139-2144. Another approach uses exponential ensemble mutagenesis to produce libraries of enzyme or other pathway variants that are, e.g., selected for an ability to catalyze a biosynthetic reaction relevant to producing an unnatural amino acid (or a new synthetase). In this approach, small groups of residues in a sequence of interest are randomized in parallel to identify, at each altered position, amino acids which lead to functional proteins. Examples of such procedures, which can be adapted to the present invention to produce new enzymes for the production of unnatural amino acids (or new synthetases) are found in Delegrave and Youvan (1993) Biotechnology Research 11:1548-1552. In yet another approach, random or semi-random mutagenesis using doped or degenerate oligonucleotides for enzyme and/or pathway component engineering can be used, e.g., by using the general mutagenesis methods of e.g., Arkin and Youvan (1992) “Optimizing nucleotide mixtures to encode specific subsets of amino acids for semi-random mutagenesis” Biotechnology 10:297-300; or Reidhaar-Olson et al. (1991) “Random mutagenesis of protein sequences using oligonucleotide cassettes” Methods Enzymol. 208:564-86. Yet another approach, often termed a “non-stochastic” mutagenesis, which uses polynucleotide reassembly and site-saturation mutagenesis can be used to produce enzymes and/or pathway components, which can then be screened for an ability to perform one or more synthetase or biosynthetic pathway function (e.g., for the production of unnatural amino acids in vivo). See, e.g., Short “Non-Stochastic Generation of Genetic Vaccines and Enzymes” WO 00/46344.

An alternative to such mutational methods involves recombining entire genomes of organisms and selecting resulting progeny for particular pathway functions (often referred to as “whole genome shuffling”). This approach can be applied to the present invention, e.g., by genomic recombination and selection of an organism (e.g., an E. coli or other cell) for an ability to produce an unnatural amino acid (or intermediate thereof). For example, methods taught in the following publications can be applied to pathway design for the evolution of existing and/or new pathways in cells to produce unnatural amino acids in vivo: Patnaik et al. (2002) “Genome shuffling of lactobacillus for improved acid tolerance” Nature Biotechnology, 20(7): 707-712; and Zhang et al. (2002) “Genome shuffling leads to rapid phenotypic improvement in bacteria” Nature 415: 644-646.

Other techniques for organism and metabolic pathway engineering, e.g., for the production of desired compounds are also available and can also be applied to the production of unnatural amino acids. Examples of publications teaching useful pathway engineering approaches include: Nakamura and White (2003) “Metabolic engineering for the microbial production of 1,3 propanediol” Curr. Opin. Biotechnol. 14(5):454-9; Berry et al. (2002) “Application of Metabolic Engineering to improve both the production and use of Biotech Indigo” J. Industrial Microbiology and Biotechnology 28:127-133; Banta et al. (2002) “Optimizing an artificial metabolic pathway: Engineering the cofactor specificity of Corynebacterium 2,5-diketo-D-gluconic acid reductase for use in vitamin C biosynthesis” Biochemistry 41:6226-36; Selivonova et al. (2001) “Rapid Evolution of Novel Traits in Microorganisms” Applied and Environmental Microbiology 67:3645, and many others.

Regardless of the method used, typically, the unnatural amino acid produced with an engineered biosynthetic pathway of the invention is produced in a concentration sufficient for efficient protein biosynthesis, e.g., a natural cellular amount, but not to such a degree as to significantly affect the concentration of other cellular amino acids or to exhaust cellular resources. Typical concentrations produced in vivo in this manner are about 10 mM to about 0.05 mM. Once a cell is engineered to produce enzymes desired for a specific pathway and an unnatural amino acid is generated, in vivo selections are optionally used to further optimize the production of the unnatural amino acid for both ribosomal protein synthesis and cell growth.

Mutagenesis and Other Molecular Biology Techniques

Polynucleotides and polypeptides of the invention and used in the invention can be manipulated using molecular biological techniques. General texts which describe molecular biological techniques include Berger and Kimmel, Guide to Molecular Cloning Techniques, Methods in Enzymology volume 152 Academic Press, Inc., San Diego, Calif.; Sambrook et al., Molecular Cloning—A Laboratory Manual (3rd Ed.), Vol. 1-3, Cold Spring Harbor Laboratory, Cold Spring Harbor, N.Y., 2001; and Current Protocols in Molecular Biology, F. M. Ausubel et al., eds., Current Protocols, a joint venture between Greene Publishing Associates, Inc. and John Wiley & Sons, Inc., (supplemented through 2005)). These texts describe mutagenesis, the use of vectors, promoters and many other relevant topics related to, e.g., the generation of nucleic acids including genes that include selector codons for production of proteins that include unnatural amino acids and to generation of orthogonal tRNAs, orthogonal synthetases, and pairs thereof.

Various types of mutagenesis are optionally used in the invention, e.g., to insert selector codons that encode an unnatural amino acid in a protein of interest into a nucleic acid (e.g., into a DNA that encodes an RNA that is to be translated to produce the protein). They include, but are not limited to, site-directed mutagenesis, random point mutagenesis, homologous recombination, DNA shuffling or other recursive mutagenesis methods, chimeric construction, mutagenesis using uracil containing templates, oligonucleotide-directed mutagenesis, phosphorothioate-modified DNA mutagenesis, mutagenesis using gapped duplex DNA or the like, or any combination thereof. Additional suitable methods include point mismatch repair, mutagenesis using repair-deficient host strains, restriction-selection and restriction-purification, deletion mutagenesis, mutagenesis by total gene synthesis, double-strand break repair, and the like.

Host cells are genetically engineered (e.g., transformed, transduced or transfected) with a relevant nucleic acid, e.g., a nucleic acid encoding an O-tRNA, O-RS, or a protein of interest including a selector codon, e.g., in a cloning vector or an expression vector. For example, the coding regions for the orthogonal tRNA, the orthogonal tRNA synthetase, and the protein to be derivatized are operably linked to gene expression control elements that are functional in the desired host cell. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both (e.g., shuttle vectors) and selection markers for both prokaryotic and eukaryotic systems. Vectors are suitable for replication and/or integration in prokaryotes, eukaryotes, or preferably both. See Giliman and Smith (1979) Gene 8:81; Roberts et al. (1987) Nature 328:731; Schneider et al. (1995) Protein Expr. Purif. 6435:10; Ausubel, Sambrook, Berger (all supra). The vector can be, for example, in the form of a plasmid, a bacterium, a virus, a naked polynucleotide, or a conjugated polynucleotide. The vectors are introduced into cells and/or microorganisms by standard methods including electroporation (From et al. (1985) Proc. Natl. Acad. Sci. USA 82:5824, infection by viral vectors, high velocity ballistic penetration by small particles with the nucleic acid either within the matrix of small beads or particles or on the surface (Klein et al. (1987) Nature 327:70-73), and/or the like.

A catalog of bacteria and bacteriophages useful for cloning is provided, e.g., by the ATCC, e.g., The ATCC Catalogue of Bacteria and Bacteriophage (1996) Ghema et al. (eds.) published by the ATCC. Additional basic procedures for sequencing, cloning and other aspects of molecular biology and underlying theoretical considerations are also found in Sambrook (supra), Ausubel (supra), and in Watson et al. (1992) Recombinant DNA Second Edition, Scientific American Books (New York). In addition, essentially any nucleic acid (and virtually any labeled nucleic acid, whether standard or non-standard) can be custom or standard ordered from any of a variety of commercial sources, such as the Midland Certified Reagent Company (Midland, Tex.; available on the World Wide Web at mcrc.com), The Great American Gene Company (Ramona, Calif.; available on the World Wide Web at genco.com), ExpressGen Inc. (Chicago, Ill.; available on the World Wide Web at expressgen.com), Operon Technologies Inc. (Alameda, Calif.) and many others.

The engineered host cells can be cultured in conventional nutrient media modified as appropriate for such activities as, for example, screening steps, activating promoters or selecting transformants. These cells can optionally be cultured into transgenic organisms. Other useful references, e.g. for cell isolation and culture (e.g., for subsequent nucleic acid isolation) include Freshney (2000) Culture of Animal Cells, a Manual of Basic Technique, fourth edition, Wiley-Liss, New York and the references cited therein; Higgins and Hames (eds) (1999) Protein Expression: A Practical Approach, Practical Approach Series, Oxford University Press; Shuler et al. (eds) (1994) Baculovirus Expression Systems and Biopesticides, Wiley-Liss; Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg New York) and Atlas and Parks (eds.) The Handbook of Microbiological Media (1993) CRC Press, Boca Raton, Fla.

Methods for Producing Labeled Proteins and Resulting Compositions

As noted, one aspect of the invention provides methods for producing a spectroscopically labeled protein. One general class of embodiments provides methods in which a nucleic acid that encodes the protein is translated in a translation system. The nucleic acid includes a selector codon. The translation system includes an orthogonal tRNA (O-tRNA) that recognizes the selector codon, an unnatural amino acid comprising a spectroscopic label, and an orthogonal aminoacyl tRNA synthetase (O-RS) that preferentially aminoacylates the O-tRNA with the unnatural amino acid. The unnatural amino acid is incorporated into the protein as it is translated in the translation system, thereby producing the spectroscopically labeled protein. Exemplary translation systems including O-tRNA/O-RS pairs, exemplary selector codons, and exemplary unnatural amino acids have been described above.

Another general class of embodiments provides methods in which a nucleic acid that encodes the protein is translated in a translation system. The nucleic acid includes a selector codon for incorporating an unnatural amino acid at a specific position in the protein. The translation system includes an orthogonal tRNA (O-tRNA) that recognizes the selector codon, the unnatural amino acid, and an orthogonal aminoacyl tRNA synthetase (O-RS) that preferentially aminoacylates the O-tRNA with the unnatural amino acid. The unnatural amino acid is incorporated into the protein as it is translated, thereby producing a translated protein comprising the unnatural amino acid at the specific position. A spectroscopic label is attached (e.g., covalently attached) to the unnatural amino acid in the translated protein, thereby producing the spectroscopically labeled protein. The translated protein is optionally purified from the translation system prior to attachment of the spectroscopic label. Exemplary translation systems including O-tRNA/O-RS pairs, exemplary selector codons, and exemplary unnatural amino acids have been described above.

The unnatural amino acid can be essentially any unnatural amino acid to which a spectroscopic label can be attached. Suitable chemically reactive unnatural amino acids include, but are not limited to, a keto amino acid, p-acetyl-L-phenylalanine, m-acetyl-L-phenylalanine, O-allyl-L-tyrosine, O-(2-propynyl)-L-tyrosine, p-ethylthiocarbonyl-L-phenylalanine, p-(3-oxobutanoyl)-L-phenylalanine, and an amino acid that can be photocrosslinked, such as p-azido-L-phenylalanine and p-benzoyl-L-phenylalanine. See, e.g., Chin et al. (2002) JACS 124:9026-7, Chin et al. (2002) PNAS 99:11020-4, and Wang and Schultz (2004) Angew. Chem. Int. Ed. 43:2-43, and references therein.

The spectroscopic label can be covalently or noncovalently attached to the unnatural amino acid by any of a variety of techniques known in the art. Typically, the spectroscopic label is functionalized for attachment to a chemically reactive unnatural amino acid. For example, keto amino acids in which the side chain comprises a carbonyl group can participate in a large number of reactions from addition and decarboxylation reactions to aldol condensations, e.g., to be selectively modified with hydrazide and hydroxylamine derivatives of spectroscopic labels. See, e.g., U.S. patent application Ser. No. 10/530,421 by Schultz et al. entitled “Site Specific Incorporation of Keto Amino Acids into Proteins,” which describes inter alia covalent attachment of a fluorophore to an unnatural amino acid via reaction of fluorescein hydrazide with p-acetyl-L-phenylalanine. As another example, a spin-label can be attached to an unnatural amino acid having a free thiol group by reacting the thiol with (1-Oxyl-2,2,5,5-tetramethylpyrroline-3-methyl) methanethiosulfonate (available from, e.g., Reanal (Budapest)). As yet another example, a spin-label (or other spectroscopic label) can be attached to an unnatural amino acid by reaction of the unnatural amino acid with an oxime, hydrazine, hydrazide, allyl, or phosphine derivative of the label (e.g., an oxime, hydrazine, hydrazide, allyl, or phosphine derivative of TEMPO). See, e.g., Saxon et al. (2000) “A ‘Traceless’ Staudinger ligation for chemoselective synthesis of amide bonds” Org. Letters, 2:2141-3 and Kohn and Breinbauer (2004) “The Staudinger ligation—A gift to chemical biology” R. Angew Chem Int Ed Engl. 43:3106-16. For example, a phosphine derivative of TEMPO (or another spectroscopic label) can be reacted with p-azido-L-phenylalanine, or an oxime, hydrazine, or hydrazide derivative of TEMPO (or another spectroscopic label) can be reacted with p-acetyl-L-phenylalanine or m-acetyl-L-phenylalanine. Similarly, 4-amino-TEMPO can be reacted with p-acetyl-L-phenylalanine or m-acetyl-L-phenylalanine to attach a TEMPO spin-label to either of these unnatural amino acids. A wide variety of such functionalized spectroscopic labels are commercially available and/or can be readily synthesized by one of skill in the art. Reactive and commercially available spin-label compounds, for example, include, but are not limited to, (1-oxyl-2,2,5,5-tetramethylpyrroline-3-methyl) methanethiosulfonate, 4-amino-2,2,6,6-tetramethylpiperidine-1-oxyl, 4-isothiocyanato-2,2,6,6-tetramethylpiperidine 1-oxyl, 3-carbamoyl-2,2,5,5-tetramethyl-3-pyrrolin-1-oxyl, 4-(2-bromoacetamido)-2,2,6,6-tetramethylpiperidine-1-oxyl, 4-(2-iodoacetamido)-2,2,6,6-tetramethylpiperidine-1-oxyl, 4-cyano-2,2,6,6-tetramethylpiperidine-1-oxyl, 4-maleimido-2,2,6,6-tetramethylpiperidine-1-oxyl, 4-oxo-2,2,6,6-tetramethylpiperidine-1-oxyl, and 4-carboxy-2,2,6,6-tetramethylpiperidine 1-oxyl.

Proteins produced by any of the methods herein form another feature of the invention, e.g., site-specific spectroscopically labeled proteins. Optionally, a protein of the invention will include a post-translational modification. An excipient (e.g., a pharmaceutically acceptable excipient), or more typically, an appropriate solution (containing, e.g., one or more buffers, salts, detergents, or the like) can also be present with the protein.

It is worth noting that the methods for producing spectroscopically labeled proteins provide the ability to synthesize proteins that comprise spectroscopically labeled unnatural amino acids in large useful quantities. Thus, in one aspect, a composition is provided that includes, e.g., at least 10 micrograms, at least 50 micrograms, at least 75 micrograms, at least 100 micrograms, at least 200 micrograms, at least 250 micrograms, at least 500 micrograms, at least 1 milligram, at least 10 milligrams, at least 50 milligrams, or at least 100 milligrams or more of a protein that comprises a spectroscopically labeled unnatural amino acid (or multiple unnatural amino acids), or an amount that can be achieved with in vivo protein production methods (details on recombinant protein production and purification are provided herein). In another aspect, the protein is optionally present in the composition at a concentration of, e.g., at least 10 micrograms of protein per liter, at least 50 micrograms of protein per liter, at least 75 micrograms of protein per liter, at least 100 micrograms of protein per liter, at least 200 micrograms of protein per liter, at least 250 micrograms of protein per liter, at least 500 micrograms of protein per liter, at least 1 milligram of protein per liter, or at least 10 milligrams of protein per liter or more, in, e.g., a cell lysate, a buffer, a pharmaceutical buffer, or other liquid suspension (e.g., in a volume of, e.g., anywhere from about 1 nL to about 100 L). The production of large quantities (e.g., greater that that typically possible with other methods, e.g., in vitro translation) of a protein in a cell including at least one spectroscopically labeled unnatural amino acid is a feature of the invention.

In one aspect of the invention, a composition includes at least one protein with at least one, and optionally, at least two, at least three, at least four, at least five, at least six, at least seven, at least eight, at least nine, or at least ten or more unnatural amino acids, e.g., spectroscopically labeled unnatural amino acids and/or other unnatural amino acids. The unnatural amino acids can be the same or different, e.g., there can be 1, 2, 3, 4, 5, 6, 7, 8, 9, or 10 or more different sites in the protein that comprise 1, 2, 3, 4, 5, 6, 7, 8, 9, or 10 or more different unnatural amino acids. In another aspect, a composition includes a protein with at least one, but fewer than all, of a particular amino acid present in the protein substituted with the spectroscopically labeled unnatural amino acid. For a given protein with more than one unnatural amino acid, the unnatural amino acids can be identical or different (e.g., the protein can include two or more different types of unnatural amino acids, or can include two of the same unnatural amino acid). For a given protein with more than two unnatural amino acids, the unnatural amino acids can be the same, different or a combination of a multiple unnatural amino acid of the same kind with at least one different unnatural amino acid.

Essentially any protein (or portion thereof) that includes an unnatural amino acid, or that encodes multiple different unnatural amino acids (and any corresponding coding nucleic acid, e.g., which includes one or more selector codons), can be produced using the compositions and methods herein. No attempt is made to identify the hundreds of thousands of known proteins, any of which can be modified to include one or more unnatural amino acid, e.g., by tailoring any available mutation methods to include one or more appropriate selector codon in a relevant translation system. Common sequence repositories for known proteins include GenBank EMBL, DDBJ and the NCBI. Other repositories can easily be identified by searching the internet.

Typically, the proteins are, e.g., at least 60%, at least 70%, at least 75%, at least 80%, at least 90%, at least 95%, or at least 99% or more identical to any available protein (e.g., a therapeutic protein, a diagnostic protein, an industrial enzyme, or a domain or other portion thereof, and the like), and they comprise one or more unnatural amino acid. Essentially any protein whose structure is of interest can be modified to include a spectroscopically labeled unnatural amino acid. Examples of therapeutic, diagnostic, and other proteins that can be modified to comprise one or more spectroscopically labeled unnatural amino acids can be found, but are not limited to, those in International Application Number PCT/US2004/011786, filed Apr. 16, 2004, entitled “Expanding the Eukaryotic Genetic Code;” and, WO 2002/085923, entitled “In vivo incorporation of unnatural amino acids.” Examples of therapeutic, diagnostic, and other proteins that can be modified to comprise one or more spectroscopically labeled unnatural amino acids include, but are not limited to, e.g., Alpha-1 antitrypsin, Angiostatin, Antihemolytic factor, antibodies (further details on antibodies are found below), Apolipoprotein, Apoprotein, Atrial natriuretic factor, Atrial natriuretic polypeptide, Atrial peptides, C—X—C chemokines (e.g., T39765, NAP-2, ENA-78, Gro-a, Gro-b, Gro-c, IP-10, GCP-2, NAP-4, SDF-1, PF4, MIG), Calcitonin, CC chemokines (e.g., Monocyte chemoattractant protein-1, Monocyte chemoattractant protein-2, Monocyte chemoattractant protein-3, Monocyte inflammatory protein-1 alpha, Monocyte inflammatory protein-1 beta, RANTES, I309, R83915, R91733, HCC1, T58847, D31065, T64262), CD40 ligand, C-kit Ligand, Collagen, Colony stimulating factor (CSF), Complement factor 5a, Complement inhibitor, Complement receptor 1, cytokines, (e.g., epithelial Neutrophil Activating Peptide-78, GROα/MGSA, GROβ, GROγ, MIP-1α, MIP-1δ, MCP-1), Epidermal Growth Factor (EGF), Erythropoietin (“EPO”), Exfoliating toxins A and B, Factor IX, Factor VII, Factor VIII, Factor X, Fibroblast Growth Factor (FGF), Fibrinogen, Fibronectin, G-CSF, GM-CSF, Glucocerebrosidase, Gonadotropin, growth factors, Hedgehog proteins (e.g., Sonic, Indian, Desert), Hemoglobin, Hepatocyte Growth Factor (HGF), Hirudin, Human serum albumin, Insulin, Insulin-like Growth Factor (IGF), interferons (e.g., IFN-α, IFN-β, IFN-γ), interleukins (e.g., IL-1, IL-2, IL-3, IL-4, IL-5, IL-6, IL-7, IL-8, IL-9, IL-10, IL-1, IL-12, etc.), Keratinocyte Growth Factor (KGF), Lactoferrin, leukemia inhibitory factor, Luciferase, Neurturin, Neutrophil inhibitory factor (NIF), oncostatin M, Osteogenic protein, Parathyroid hormone, PD-ECSF, PDGF, peptide hormones (e.g., Human Growth Hormone), Pleiotropin, Protein A, Protein G, Pyrogenic exotoxins A, B, and C, Relaxin, Renin, SCF, Soluble complement receptor I, Soluble I-CAM 1, Soluble interleukin receptors (IL-1, 2, 3, 4, 5, 6, 7, 9, 10, 11, 12, 13, 14, 15), Soluble TNF receptor, Somatomedin, Somatostatin, Somatotropin, Streptokinase, Superantigens, i.e., Staphylococcal enterotoxins (SEA, SEB, SEC1, SEC2, SEC3, SED, SEE), Superoxide dismutase (SOD), Toxic shock syndrome toxin (TSST-1), Thymosin alpha 1, Tissue plasminogen activator, Tumor necrosis factor beta (TNF beta), Tumor necrosis factor receptor (TNFR), Tumor necrosis factor-alpha (TNF alpha), Vascular Endothelial Growth Factor (VEGEF), Urokinase and many others.

One class of proteins that can be made using the compositions and methods for in vivo incorporation of spectroscopically labeled unnatural amino acids described herein includes transcriptional modulators or a portion thereof. Example transcriptional modulators include genes and transcriptional modulator proteins that modulate cell growth, differentiation, regulation, or the like. Transcriptional modulators are found in prokaryotes, viruses, and eukaryotes, including fungi, plants, yeasts, insects, and animals, including mammals, providing a wide range of therapeutic targets. It will be appreciated that expression and transcriptional activators regulate transcription by many mechanisms, e.g., by binding to receptors, stimulating a signal transduction cascade, regulating expression of transcription factors, binding to promoters and enhancers, binding to proteins that bind to promoters and enhancers, unwinding DNA, splicing pre-mRNA, polyadenylating RNA, and degrading RNA.

Another class of proteins of the invention (e.g., proteins with one or more spectroscopically labeled unnatural amino acids) include expression activators such as cytokines, inflammatory molecules, growth factors, their receptors, and oncogene products, e.g., interleukins (e.g., IL-1, IL-2, IL-8, etc.), interferons, FGF, IGF-I, IGF-II, FGF, PDGF, TNF, TGF-α, TGF-β, EGF, KGF, SCF/c-Kit, CD40L/CD40, VLA-4NCAM-1, ICAM-1/LFA-1, and hyalurin/CD44; signal transduction molecules and corresponding oncogene products, e.g., Mos, Ras, Raf, and Met; and transcriptional activators and suppressors, e.g., p53, Tat, Fos, Myc, Jun, Myb, Rel, and steroid hormone receptors such as those for estrogen, progesterone, testosterone, aldosterone, the LDL receptor ligand and corticosterone.

Enzymes (e.g., industrial enzymes) or portions thereof with at least one spectroscopically labeled unnatural amino acid are also provided by the invention. Examples of enzymes include, but are not limited to, e.g., amidases, amino acid racemases, acylases, dehalogenases, dioxygenases, diarylpropane peroxidases, epimerases, epoxide hydrolases, esterases, isomerases, kinases, glucose isomerases, glycosidases, glycosyl transferases, haloperoxidases, monooxygenases (e.g., p450s), lipases, lignin peroxidases, nitrile hydratases, nitrilases, proteases, phosphatases, subtilisins, transaminase, and nucleases.

Many of these proteins are commercially available (see, e.g., the Sigma BioSciences 2004 catalog and price list), and the corresponding protein sequences and genes and, typically, many variants thereof, are well-known (see, e.g., Genbank). Any of them can be modified by the insertion of one or more spectroscopically labeled unnatural amino acid or other unnatural amino acid according to the invention, e.g., to facilitate determination of the protein's structure and/or properties.

A variety of other proteins can also be modified to include one or more spectroscopically labeled unnatural amino acid. For example, the invention can include substituting one or more natural amino acids in one or more vaccine proteins with a spectroscopically labeled unnatural amino acid, e.g., in proteins from infectious fungi, e.g., Aspergillus, Candida species; bacteria, particularly E. coli, which serves a model for pathogenic bacteria, as well as medically important bacteria such as Staphylococci (e.g., aureus), or Streptococci (e.g., pneumoniae); protozoa such as sporozoa (e.g., Plasmodia), rhizopods (e.g., Entamoeba) and flagellates (Trypanosoma, Leishmania, Trichomonas, Giardia, etc.); viruses such as (+) RNA viruses (examples include Poxviruses e.g., vaccinia; Picomaviruses, e.g. polio; Togaviruses, e.g., rubella; Flaviviruses, e.g., HCV; and Coronaviruses), (−) RNA viruses (e.g., Rhabdoviruses, e.g., VSV; Paramyxovimses, e.g., RSV; Orthomyxovimses, e.g., influenza; Bunyaviruses; and Arenaviruses), dsDNA viruses (Reoviruses, for example), RNA to DNA viruses, i.e., Retroviruses, e.g., HIV and HTLV, and certain DNA to RNA viruses such as Hepatitis B.

Agriculturally related proteins such as insect resistance proteins (e.g., the Cry proteins), starch and lipid production enzymes, plant and insect toxins, toxin-resistance proteins, Mycotoxin detoxification proteins, plant growth enzymes (e.g., ribulose 1,5-bisphosphate carboxylase/oxygenase, “RUBISCO”), lipoxygenase (LOX), and phosphoenolpyruvate (PEP) carboxylase are also suitable targets for spectroscopically labeled unnatural amino acid or other unnatural amino acid modification.

In certain embodiments, the protein of interest (or portion thereof) in the methods and/or compositions of the invention is encoded by a nucleic acid. Typically, the nucleic acid comprises at least one selector codon, at least two selector codons, at least three selector codons, at least four selector codons, at least five selector codons, at least six selector codons, at least seven selector codons, at least eight selector codons, at least nine selector codons, or ten or more selector codons.

Nucleic acids (e.g., genes) coding for proteins of interest can be mutagenized using methods well-known to one of skill in the art and described herein under “Mutagenesis and Other Molecular Biology Techniques” to include, e.g., one or more selector codon for the incorporation of a spectroscopically labeled unnatural amino acid. For example, a nucleic acid for a protein of interest is mutagenized to include one or more selector codon, providing for the insertion of the one or more spectroscopically labeled unnatural amino acids. The invention includes any such variant, e.g., mutant, versions of any protein, e.g., including at least one spectroscopically labeled unnatural amino acid. Similarly, the invention also includes corresponding nucleic acids, i.e., any nucleic acid with one or more selector codon that encodes one or more spectroscopically labeled unnatural amino acid.

To make a protein that includes a spectroscopically labeled unnatural amino acid, one can use host cells and organisms that are adapted for the in vivo incorporation of the spectroscopically labeled unnatural amino acid via orthogonal tRNA/RS pairs. Host cells are genetically engineered (e.g., transformed, transduced or transfected) with one or more vectors that express the orthogonal tRNA, the orthogonal tRNA synthetase, and a vector that encodes the protein to be derivatized. Each of these components can be on the same vector, or each can be on a separate vector, or two components can be on one vector and the third component on a second vector. The vector can be, for example, in the form of a plasmid, a bacterium, a virus, a naked polynucleotide, or a conjugated polynucleotide.

Protein Spectroscopy

As noted above, site-specific, efficient incorporation of spectroscopically labeled unnatural amino acids, or of unnatural amino acids to which a spectroscopic label is then attached, into proteins facilitates studies of the proteins by spectroscopic techniques, including, but not limited to, NMR spectroscopy, EPR spectroscopy, X-ray spectroscopy, UV spectrometry, mass spectroscopy, fluorescence spectroscopy, and vibrational (e.g., infrared or Raman) spectroscopy.

Methods Using Spectroscopically Labeled Proteins

Also as noted, one general class of embodiments provides methods for producing a spectroscopically labeled protein, in which methods a nucleic acid that encodes the protein is translated in a translation system. The nucleic acid includes a selector codon. The translation system includes an orthogonal tRNA (O-tRNA) that recognizes the selector codon, an unnatural amino acid comprising a spectroscopic label, and an orthogonal aminoacyl tRNA synthetase (O-RS) that preferentially aminoacylates the O-tRNA with the unnatural amino acid. The unnatural amino acid is incorporated into the protein as it is translated, thereby producing the spectroscopically labeled protein.

In this class of embodiments, the methods optionally include subjecting the spectroscopically labeled protein to a spectroscopic technique, including, but not limited to, NMR spectroscopy, EPR spectroscopy, UV spectrometry, X-ray spectroscopy (e.g., for detection of radiation), mass spectroscopy, fluorescence spectroscopy, or vibrational (e.g., infrared or Raman) spectroscopy. As just one example, in one embodiment, the spectroscopically labeled protein comprises a ¹⁵N isotope, and the spectroscopic technique comprises a solvent-exposed amine transverse relaxation optimized spectroscopy (SEA-TROSY) experiment. As another specific example, the spectroscopically labeled protein can comprise a ¹⁹F isotope, and the spectroscopic technique can comprise a one-dimensional non-proton NMR experiment (e.g., to study conformational changes, ligand binding, or the like). Many other spectroscopic techniques (e.g., NMR techniques such as NOESY, HSQC, HSQC-NOESY, TROSY, SEA-TROSY, and TROSY-HSQC) are well known in the art and can be adapted for use in the methods of the invention, and many such techniques are described below in the section entitled “Spectroscopic Techniques.”

Another general class of embodiments provides methods for producing a spectroscopically labeled protein, where the spectroscopic label is attached to an unnatural amino acid after the unnatural amino acid is incorporated into the protein. In the methods, a nucleic acid that encodes the protein is translated in a translation system. The nucleic acid includes a selector codon for incorporating an unnatural amino acid at a specific position in the protein. The translation system includes an orthogonal tRNA (O-tRNA) that recognizes the selector codon, the unnatural amino acid, and an orthogonal aminoacyl tRNA synthetase (O-RS) that preferentially aminoacylates the O-tRNA with the unnatural amino acid. The unnatural amino acid is incorporated into the protein as it is translated, thereby producing a translated protein comprising the unnatural amino acid at the specific position. A spectroscopic label is attached (e.g., covalently attached) to the unnatural amino acid in the translated protein, thereby producing the spectroscopically labeled protein.

In this class of embodiments, the methods optionally include subjecting the spectroscopically labeled protein to a spectroscopic technique, including, but not limited to, NMR spectroscopy, EPR spectroscopy, UV spectrometry, X-ray spectroscopy (e.g., for detection of radiation), mass spectroscopy, fluorescence spectroscopy, or vibrational (e.g., infrared or Raman) spectroscopy. As just one example, in one embodiment, the spectroscopic technique is NMR spectroscopy, and the spectroscopic label comprises a chelator and a paramagnetic metal associated with the chelator. As another specific example in which the spectroscopic technique is NMR spectroscopy, the spectroscopic label comprises a spin-label. When NMR analysis of a spin-labeled protein is performed, optionally an NMR experiment is performed on the spectroscopically labeled protein and a first set of data is collected, and then the spectroscopically labeled protein is reduced (e.g., by addition of a reducing agent such as ascorbic acid) to provide a reduced form of the spectroscopically labeled protein, an NMR experiment is performed on the reduced form of the spectroscopically labeled protein, and a second set of data is collected to provide a reference spectrum. Many other spectroscopic techniques (e.g., NMR techniques) are well known in the art and can be adapted for use in the methods of the invention, and many such techniques are described below in the section entitled “Spectroscopic Techniques.”

In either general class of embodiments, the spectroscopic technique is optionally performed on the spectroscopically labeled protein in vivo, e.g., in intact cells, intact tissue, or the like. Alternatively, the spectroscopic technique can be performed on the spectroscopically labeled protein in vitro, e.g., in a cellular extract, on a purified or partially purified protein, or the like.

In either general class of embodiments, the spectroscopic technique can be used, e.g., to obtain information about the structure, function, abundance, and/or dynamics of the protein, e.g., two-dimensional structure, three-dimensional structure, conformational changes, ligand binding, catalytic mechanism, protein folding, protein concentration, and/or the like. For example, in one class of embodiments, the methods include subjecting the spectroscopically labeled protein to a spectroscopic technique and generating information regarding one or more changes in structure or dynamics of the spectroscopically labeled protein. In some embodiments, the methods include analyzing an interaction between the spectroscopically labeled protein and a ligand or substrate. The interaction can include, e.g., a change in conformation in the spectroscopically labeled protein, binding of a ligand to a specific site near the spectroscopic label, and/or a catalytic reaction performed by the spectroscopically labeled protein.

Methods for NMR Resonance Assignment Using Isotopically Labeled Proteins

Assignment of resonances to particular amino acids in a protein of interest is a key step in NMR studies. Typically, a resonance (an individual signal in an NMR spectrum) is assigned to a particular atom (e.g., the alpha carbon of a particular amino acid) or group of indistinguishable atoms (e.g., the three protons of a methyl group).

Site-specific isotopic labeling of a protein, e.g., using an unnatural amino acid containing an NMR active isotope, can greatly simplify the process of resonance assignment, whether many, a few, or even only one resonance is being assigned. For example, in NMR studies of a protein's three-dimensional structure, isotopic labeling of the protein can aid assignment of relevant resonances to their corresponding amino acids, e.g., for resonances difficult to assign by other techniques. As another example, assigning only a single residue (or a small number of residues) at or near an active site, ligand binding site, protein-protein interface, or the like is sometimes desirable, in which case isotopic labeling of the relevant residue(s) can facilitate detailed NMR analysis of even very large proteins.

Accordingly, one general class of embodiments provides methods for assigning NMR resonances to one or more amino acid residues in a protein of interest. In the methods, an unnatural amino acid comprising an NMR active isotope is provided and incorporated, producing an isotopically-labeled protein of interest, in a translation system. The translation system includes a nucleic acid encoding the protein of interest and comprising at least one selector codon for incorporating the unnatural amino acid at a specific site in the protein (e.g., at a selected position in the amino acid sequence of the protein), an orthogonal tRNA (O-tRNA) that recognizes the selector codon, and an orthogonal aminoacyl tRNA synthetase (O-RS) that preferentially aminoacylates the O-tRNA with the unnatural amino acid. An NMR experiment is performed on the isotopically labeled protein, and data generated due to an interaction between the NMR active isotope of the unnatural amino acid and a proximal atom is analyzed, whereby one or more NMR resonances are assigned to one or more amino acid residues in the protein.

Exemplary translation systems including O-tRNA/O-RS pairs, exemplary selector codons, and exemplary unnatural amino acids have been described above. The NMR active isotope on the unnatural amino acid can be essentially any suitable isotope, including, e.g. ²H, ¹³C, ¹⁵N, ³H, ⁷Li, ¹³B, ¹⁴N, ¹⁷O, ¹⁹F, ²³Na, ²⁷Al, ²⁹Si, ³¹P, ³⁵Cl, ³⁷Cl, ³⁹K, ⁵⁹Co, ⁷⁷Se, ⁸¹Br, ¹¹³Cd, ¹¹⁹Sn, and ¹⁹⁵Pt.

A variety of NMR techniques are well known in the art and can be applied to the methods of the present invention. For example, the NMR experiment can be an HSQC experiment, a TROSY experiment, a SEA-TROSY experiment, a TROSY-HSQC experiment, a NOESY experiment, an HSQC-NOESY experiment, or any of the other suitable experiments known in the art and/or described below in the section entitled “Spectroscopic Techniques.”

The specific site at which the isotopically labeled unnatural amino acid is incorporated can be essentially any site which is of interest. For example, the specific site of the unnatural amino acid can comprise an active site or ligand binding site of the protein, or it can comprise a site proximal to an active site or ligand binding site of the protein.

The NMR experiment can be performed in vivo or in vitro. Thus, for example, data can be collected in vivo on the isotopically labeled protein, on a cellular extract comprising the isotopically labeled protein, or on a purified or substantially purified isotopically labeled protein.

A related general class of embodiments also provides methods for resonance assignment. In these methods for assigning an NMR resonance to an amino acid residue occupying a specific position in a protein of interest, the methods include providing a first sample comprising the protein. In this first sample, the protein comprises, at the specific position, an amino acid residue comprising an NMR active isotope. An NMR experiment is performed on the first sample and a first set of data is collected. A second sample comprising the protein is also provided, in which the protein comprises, at the specific position, an unnatural amino acid lacking the NMR active isotope. An NMR experiment is performed on the second sample and a second set of data is collected. The first and second sets of data are compared, whereby a resonance present in the first set and not present in the second set is assigned to the amino acid residue at the specific position.

In a preferred class of embodiments, the second sample is provided by translating a nucleic acid that encodes the protein in a translation system. The nucleic acid comprises a selector codon for incorporating the unnatural amino acid at the specific position in the protein. The translation system includes an orthogonal tRNA (O-tRNA) that recognizes the selector codon, the unnatural amino acid lacking the NMR active label, and an orthogonal aminoacyl tRNA synthetase (O-RS) that preferentially aminoacylates the O-tRNA with the unnatural amino acid. The NMR active isotope can be, e.g., ¹H, ¹⁵N, ¹³C, or ¹⁹F.

These methods can be useful for, e.g., resolving ambiguities in resonance assignments, e.g., during determination of the three-dimensional structure of the protein. For example, if resonances are being assigned for a fully ¹⁵N and/or ¹³C labeled protein, the unlabeled unnatural amino acid can be incorporated into an otherwise fully labeled protein, and by the disappearance of the signal from that residue, a resonance can be assigned. For example, the ¹⁵N signal of a particular tyrosine residue could be assigned if that tyrosine is replaced by O-methyl-tyrosine not labeled with ¹⁵N, assuming that incorporation of the unnatural amino acid does not perturb the protein's structure. The methods can also be applied to ¹H spectra, partially ¹⁵N and/or ¹³C labeled proteins, and/or the like.

Essentially all of the features noted above apply to this embodiment as well, as relevant, e.g., for NMR active isotopes, composition of the translation system, NMR techniques, and the like. As for the embodiments above, the specific position at which the unnatural amino acid is incorporated can be essentially any site which is of interest in the protein.

Spectroscopic Techniques

A variety of spectroscopic techniques are known in the art and can be adapted to the methods of the present invention. Protein NMR spectroscopy, for example, is described in, e.g., Cavanagh et al. (1995) Protein NMR Spectroscopy: Principles and Practice, Academic Press; Levitt (2001) Spin Dynamics: Basics of Nuclear Magnetic Resonance, John Wiley & Sons; Evans (1995) Biomolecular NMR Spectroscopy, Oxford University Press; Wüthrich (1986) NMR of Proteins and Nucleic Acids (Baker Lecture Series), Kurt Wiley-Interscience; Neuhaus and Williamson (2000) The Nuclear Overhauser Effect in Structural and Conformational Analysis, 2nd Edition, Wiley-VCH; Macomber (1998) A Complete Introduction to Modern NMR Spectroscopy, Wiley-Interscience; Downing (2004) Protein NMR Techniques (Methods in Molecular Biology), 2nd edition, Humana Press; Clore and Gronenbom (1994) NMR of Proteins (Topics in Molecular and Structural Biology), CRC Press; Reid (1997) Protein NMR Techniques, Humana Press; Krishna and Berliner (2003) Protein NMR for the Millenium (Biological Magnetic Resonance), Kluwer Academic Publishers; Kiihne and De Groot (2001) Perspectives on Solid State NMR in Biology (Focus on Structural Biology, 1), Kluwer Academic Publishers; and Jones et al. (1993) Spectroscopic Methods and Analyses: NMR, Mass Spectrometry, and Related Techniques (Methods in Molecular Biology, Vol. 17), Humana Press.

A variety of single-dimensional (1D) and multi-dimensional (e.g., 2D, 3D and 4D) NMR spectroscopic techniques have been described, including both solution and solid-state NMR techniques. Such techniques include, e.g., 1D heteronuclear correlation experiments, 1D heteronuclear filtered experiments, COSY, NOESY, HSQC (¹H-¹⁵N heteronuclear single quantum correlation spectroscopy), HSQC-NOESY, HETCOR, TROSY (transverse relaxation optimized spectroscopy), SEA-TROSY (solvent-exposed amine transverse relaxation optimized spectroscopy), TROSY-HSQC, CRINEPT-TROSY, CRIPT-TROSY, PISEMA (polarization inversion with spin exchange at the magic angle), MAS (magic angle spinning), and MAOSS (magic angle oriented single spinning), among many others. See, e.g., the above NMR references as well as Wider (2000) BioTechniques 29:1278-1294; Pellecchia et al. (2002) Nature Rev. Drug Discov. (2002) 1:211-219; Arora and Tamm (2001) Curr. Opin. Struct. Biol. 11:540-547; Flaux et al. (2002) Nature 418:207-211; Pellecchia et al. (2001) J. Am. Chem. Soc. 123:4633-4634; and Pervushin et al. (1997) Proc. Natl. Acad. Sci. USA 94:12366-12371.

A variety of spin-labels have been described in the art, as have a number of uses for spin-labels, e.g., in NMR studies of protein structure and dynamics. For example, NMR resonances of a uniformly isotopically (for example, ¹⁵N) labeled protein that includes a spin-label will be broadened by paramagnetic relaxation enhancement dependent on the distance (˜R⁶) of the reporter group relative to the spin-label. For a protein of known structure, this method can be used for resonance assignments, especially in conjunction with amino-acid-type selectively labeled protein (similar to the technique described in Cutting et al. (2004) “NMR resonance assignment of selectively labeled proteins by the use of paramagnetic ligands” J. Biomol. NMR 30:205-10). Site-directed introduction of a spin-label into a protein as described herein can also be used to screen for ligand binding to a site near the spin-label (see e.g., the SLAPSTIC method, Jahnke et al. (2001) JACS 123:3149-50). In addition, paramagnetic relaxation enhancement by site-directed spin-labeling as described herein can provide distance restraints (e.g., long-range distance restraints) for protein structure calculations (Battiste and Wagner (2000) Biochemistry 39:5355-65). This technique can facilitate structure determination by NMR, including structure determination of large proteins, including membrane proteins. It will be evident that the unnatural amino acid comprising the spin-labeled group (whether the group is attached before or after incorporation of the amino acid into the protein) is not typically spectroscopically studied itself; it is the effect of the spin-label on other NMR active nuclei throughout the protein that is typically observed spectroscopically. Introduction of spin-labels site-specifically into proteins using unnatural amino acids, either directly via unnatural amino acids comprising spin-labels or indirectly via unnatural amino acids providing an attachment point for spin-labels, has significant advantages over current methods for introduction of spin-labels (e.g., via S—S bond formation to cysteine mutants); for example, with the methods of the invention, spin-labels can be readily incorporated at sites not occupied (or occupiable) by cysteine residues. Since spin-labels are paramagnetic in their oxidized form but lose their usefulness upon reduction, the labels are typically protected from oxidation, e.g., by attaching the spin-label to the protein in the final step before the NMR measurement of paramagnetic relaxation enhancement. A reference spectrum is typically collected on the reduced form, e.g., after addition of a reducing agent such as ascorbic acid to the NMR sample containing the spin-labeled protein.

For additional details of spin-labels and NMR, see, e.g., Jahnke (2002) “Spin labels as a tool to identify and characterize protein-ligand interactions by NMR spectroscopy” ChemBioChem 3:167-173; R. A. Dwek (1973) Monographs on Physical Biochemistry: Nuclear Magnetic Resonance (N.M.R.) in Biochemistry. Applications to enzyme systems Oxford University Press, New York; P. A. Kosen (1989) Methods Enzymol. 177:86; Hubbell (1996) “Watching proteins move using site-directed spin labeling” Structure 4:781; Hustedt and Beth (1999) “Nitroxide spin-spin interactions: Applications to Protein Structure and Dynamics” Annual Review of Biophysics and Biomolecular Structure 28:129-153; Berliner, ed. (1976) Spin Labeling: Theory and Applications New York: Academic; Berliner, ed. (1979) Spin Labeling II: Theory and Applications New York: Academic; Berliner and Reuben, eds. (1989) Biological Magnetic Resonance. Vol. VIII: Spin Labeling Theory and Applications New York: Plenum, including, e.g., Hideg and Hankovszky “Chemistry of spin-labeled amino acids and peptides. Some new mono- and bifunctionalized nitroxide free radicals” pp. 427-488; Hanson et al. (1998) “Electron spin resonance and structural analysis of water soluble, alanine-rich peptides incorporating TOAC” Mol. Phys. 95:95766; Hanson P et al. (1996) “Distinguishing helix conformations in alanine-rich peptides using the unnatural amino acid TOAC and electron spin resonance” J. Am. Chem. Soc. 118:271; Hanson et al. (1996) “ESR characterization of hexameric, helical peptides using double TOAC spin labeling” J. Am. Chem. Soc. 118:7618; Rassat and Rey (1967) Bull. Soc. Chim. France 3:815-817; Jahnke et al. (2001) J. Am. Chem. Soc. 123:3149-3150; Mchaourab et al. (1996) “Motion of spin-labeled side chains in T4 lysozyme. Correlation with protein structure and dynamics” Biochemistry 35:7692-7704; and Columbus et al. (2001) “Molecular motion of spin labeled side chains in α-helices: Analysis by variation of side chain structure” Biochemistry 40:3828-3846.

Chelators for paramagnetic metals and their uses in NMR studies have been similarly well described. They can be used, for example, for NMR protein structure refinement (Donaldson et al. (2001) “Structural characterization of proteins with an attached ATCUN motif by paramagnetic relaxation enhancement NMR spectroscopy” J. Am. Chem. Soc. 123:9843-9847 and Pintacuda et al. (2004) “Site-specific labelling with a metal chelator for protein-structure refinement” J. Biomolecular NMR 29:351-361), for resonance assignments (Pintacuda et al. (2004) “Fast structure-based assignment of ¹⁵N HSQC spectra of selectively ¹⁵N-labeled paramagnetic proteins” J. Am. Chem. Soc. 126:2963-2970), and for magnetically aligning proteins for the measurement of residual dipolar couplings (Barbieri et al. (2002) “Structure-independent cross-validation between residual dipolar couplings originating from internal and external orienting media” J. Biomolecular NMR 22:365-368 and Barbieri et al. (2002) “Paramagnetically induced residual dipolar couplings for solution structure determination of lanthanide binding proteins” J. Am. Chem. Soc. 124:5581-5587, and references therein). A reference spectrum is optionally collected on a form of the protein that includes the chelator but not the paramagnetic metal, e.g., before addition of the paramagnetic metal to the chelator.

EPR spectroscopy (electron paramagnetic resonance spectroscopy, sometimes called electron spin resonance or ESR spectroscopy) is similar to NMR, the fundamental difference being that EPR is concerned with the magnetically induced splitting of electronic spin states, while NMR describes transitions between nuclear spin states. EPR spectroscopy is similarly well described in the literature, as are UV spectrometry, X-ray spectroscopy, mass spectroscopy, fluorescence spectroscopy, and vibrational (e.g., infrared or Raman) spectroscopy. See, e.g., Weil et al. (1994) Electron Paramagnetic Resonance: Elementary Theory and Practical Applications, Wiley-Interscience; Carmona, et al. (1997) Spectroscopy of Biological Molecules: Modern Trends, Kluwer Academic Publishers; Hester et al. (1996) Spectroscopy of Biological Molecules, Special Publication Royal Society of Chemistry (Great Britain); Spiro (1987) Biological Aplications of Raman Spectroscopy, John Wiley & Sons Inc; and Jones et al. (1993) Spectroscopic Methods and Analyses: NMR, Mass Spectrometry, and Related Techniques (Methods in Molecular Biology, Vol. 17), Humana Press.

A variety of spectrometers are commercially available. For example, NMR spectrometers are available, e.g., from Varian (Palo Alto, Calif.; available on the World Wide Web at varianinc.com) and Bruker (Germany; available on the World Wide Web at bruker.com).

Protein Purification

Spectroscopic analysis of labeled proteins can be performed in vivo or in vitro, on unpurified, partially purified, or purified proteins. When purification of a spectroscopically (e.g., isotopically) labeled protein, or a protein to be so labeled, from the translation system is desired, such purification can be accomplished by any of a number of methods well known in the art, including, e.g., ammonium sulfate or ethanol precipitation, centrifugation, acid or base extraction, column chromatography, affinity column chromatography, anion or cation exchange chromatography, phosphocellulose chromatography, high performance liquid chromatography (HPLC), gel filtration, hydrophobic interaction chromatography, hydroxylapatite chromatography, lectin chromatography, gel electrophoresis, and the like.

In addition to other references noted herein, a variety of protein purification methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2nd Edition Wiley-Liss, NY; Walker (1996) The Protein Protocols Handbook Humana Press, N.J.; Harris and Angal (1990) Protein Purification Applications: A Practical Approach IRL Press at Oxford, Oxford, England; Scopes (1993) Protein Purification: Principles and Practice 3rd Edition Springer Verlag, N.Y.; Janson and Ryden (1998) Protein Purification: Principles, High Resolution Methods and Applications, Second Edition Wiley-VCH, NY; and Walker (1998) Protein Protocols on CD-ROM Humana Press, N.J.; and the references cited therein.

Well known techniques for refolding proteins can be used if necessary to obtain the active conformation of the protein when the protein is denatured during intracellular synthesis, isolation or purification. Methods of reducing, denaturing and renaturing proteins are well known to those of skill in the art (see the references above and Debinski, et al. (1993) J. Biol. Chem., 268: 14065-14070; Kreitman and Pastan (1993) Bioconjug. Chem. 4:581-585; and Buchner, et al. (1992) Anal. Biochem. 205:263-270).

The nucleotide sequence encoding the polypeptide can optionally be fused in-frame to a sequence encoding a module (e.g., a domain or tag) that facilitates purification of the polypeptide and/or facilitates association of the fusion polypeptide with a particle, a solid support or another reagent. Such modules include, but are not limited to, metal chelating peptides such as histidine-tryptophan modules that allow purification on and/or binding to immobilized metals (e.g., a hexahistidine tag), a sequence which binds glutathione (e.g., GST), a hemagglutinin (HA) tag (corresponding to an epitope derived from the influenza hemagglutinin protein; see Wilson et al. (1984) Cell 37:767), maltose binding protein sequences, the FLAG epitope utilized in the FLAGS extension/affinity purification system (Immunex Corp, Seattle, Wash.), and the like. The inclusion of a protease-cleavable polypeptide linker sequence between the purification domain and the sequence of the invention is useful to permit removal of the module following, or during, purification of the polypeptide.

### EXAMPLE

It is understood that the examples and embodiments described herein are for illustrative purposes only and that various modifications or changes in light thereof will be suggested to persons skilled in the art and are to be included within the spirit and purview of this application and scope of the appended claims. Accordingly, the following example is offered to illustrate, but not to limit the claimed invention.

### Example 1

### Site-Specific In Vivo Labeling of a Protein for NMR Studies

The following sets forth a series of experiments that demonstrate site-specific labeling of a protein for NMR. An isotopically labeled amino acid is incorporated into the protein, facilitating NMR studies of the protein (e.g., resonance assignment).

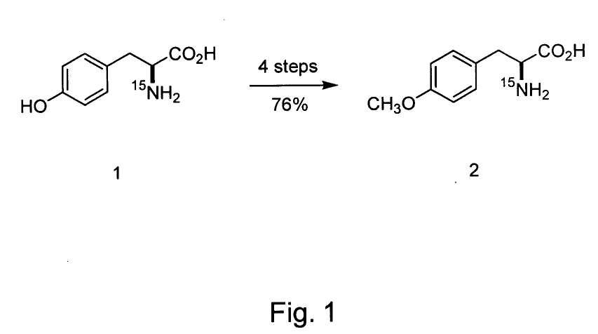

An M. jannaschii tyrosyl tRNA/tRNA-synthetase pair has been demonstrated to be orthogonal in E. coli, i.e., neither the tRNA nor the synthetase cross reacts with endogenous E. coli tRNAs or synthetases. The specificity of this and other orthogonal tRNA-synthetase pairs can be evolved to allow the selective and efficient incorporation of a number of unnatural amino acids in response to nonsense and frameshift codons, including keto, sugar, azido, alkynyl, and photocrosslinking amino acids (Alfonta et al. (2003) J. Am. Chem. Soc. 125:14662, Deiters et al. (2003) J. Am. Chem. Soc. 125:11782, Zhang et al. (2003) Biochemistry 42:6735, and Chin et al. (2002) Proc. Natl. Acad. Sci. 99:11020). In order to selectively introduce an isotopically-labeled amino acid into a protein in E. coli by this method, it must have distinct structural differences from the common 20 amino acids. This difference cannot rely on the isotope itself, since the wildtype synthetase for any particular common amino acid cannot sufficiently distinguish isotopically substituted amino acids and thus would incorporate them throughout the protein. Therefore a ¹⁵N-labeled phenylalanine derivative 2 was synthesized from commercially available material 1 in four steps and an overall yield of 76% (FIG. 1). The reaction sequence consists of a Boc-protection of the amino group (Boc₂O, Et₃N, dioxane/H₂O), simultaneous methylation of the hydroxy and the carboxy group (MeI, K₂CO₃, DMF), removal of the Boc group (HCl, MeOH), and a subsequent saponification of the ester (NaOH, MeOH/H₂O). The methoxy group is sufficient for the translational machinery of E. coli to differentiate it from phenylalanine, tyrosine, and other natural amino acids, yet it is small enough to minimize structural perturbations within the protein of interest.

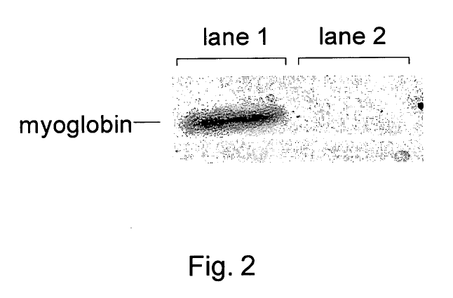

To incorporate 2 into proteins at unique sites, an orthogonal TyrRS/tRNACUA pair previously evolved in E. coli that genetically encodes p-methoxyphenylalanine was used. This tRNA synthetase pair was used to incorporate p-methoxyphenylalanine into dihydrofolate reductase with high fidelity and efficiency (Wang et al. (2001) Science 292:498). In this example, this tRNA_{CUA}/TyrRS pair is used to selectively incorporate 2 into sperm whale myoglobin, a monomeric 153-residue heme protein involved in oxygen storage in muscle that has been the focus of structural and kinetic studies over a period of decades (Reedy and Gibney (2004) Chem. Rev. 104:617 and references therein). Apo-myoglobin, which is derived from myoglobin by extracting the iron-porphyrin prosthetic group, has been widely studied as a model system for protein folding (Uzawa et al. (2004) Proc. Natl. Acad. Sci. USA 101:1171 and references therein, and Wright and Baldwin (2000) in Frontiers in Molecular Biology: Mechanisms of Protein Folding, R. Pain, ed., Oxford University Press, London, pp. 309). Myoglobin is therefore an attractive model system to take advantage of the site-specific introduction of NMR probes for future studies of protein folding. To produce site-specifically ¹⁵N-labeled myoglobin, the fourth codon (Ser4) was mutated to TAG and a C-terminal 6×His tag was added. In the presence of the mutant MjTyrRS, tRNA_{CUA}, and 2 (1 mM in liquid minimal media), full-length myoglobin was produced with a yield of 1 mg/L after purification by Ni-affinity chromatography and judged to be >90% homogeneous by SDS-Page and Gelcode Blue staining. In the absence of 2 no myoglobin was visible, revealing a fidelity for the incorporation of 2 of >99% (FIG. 2).

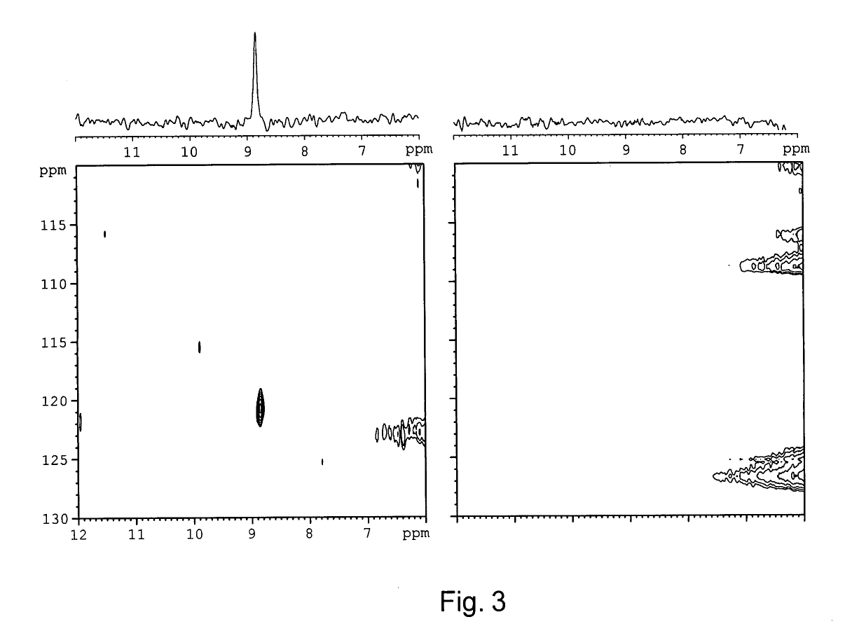

The purified protein was dialysed against 50 mM phosphate buffer (pH 5.6) and concentrated to give 0.5 mL of a 55 μM NMR sample (90%:10% H₂O/D₂O)—an amount of site-specifically labeled protein that would have been very difficult to produce by in vitro methods (Ellman et al. (1992) J. Am. Chem. Soc. 114:7959). A similar sample was prepared using non-labeledp-methoxyphenylalanine. Both samples were used in ¹H-¹⁵N HSQC experiments that were acquired with 64 t₁ increments and 512 scans per increment on a Bruker Avance 600 at 300K. The spectrum of the ¹⁵N-labeled protein shows a single amide correlation peak at 8.86 ppm (¹H chemical shift) for the amide proton and 120.6 ppm (¹⁵N chemical shift) for the amide nitrogen resonance. The same region of a ¹H-¹⁵N HSQC experiment acquired under the same conditions for the unlabeled myoglobin sample shows no correlation peak (FIG. 3).

In summary, genetically encoded isotopically-labeled amino acids can be used to obtain amounts of site-specifically labeled proteins sufficient for NMR studies. (It is worth noting that a similar labeling technique has been used for protein structure determination by x-ray crystallography, where incorporation of one or more heavy atom-containing unnatural amino acids facilitates phase determination; see U.S. Ser. No. 60/602,048.) Since our in vivo expression system uses defined minimal media, in addition to incorporation of the ¹⁵N label, fully or partially deuterated protein samples of large proteins can be produced. Additional positions in p-methoxyphenylalanine, or in other unnatural amino acids, can also be labeled, e.g., with ²H and ¹³C isotopes. The production of site-specifically labeled proteins is also be possible in yeast (Chin et al. (2003) Science 301:964) and therefore establishes a route to obtain proteins with posttranscriptional modifications. This methodology can thus enable detailed studies of larger proteins and their interactions with ligands, their conformational changes, and their mechanism of catalysis. Moreover, this in vivo labeling technique can allow in-cell NMR applications by facilitating the observation of a particular protein in the context of other macromolecules (Serber et al. (2004) J. Am. Chem. Soc. 126:7119-7125 and references therein).

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method for producing and analyzing a spectroscopically labeled protein, the method comprising:
translating a nucleic acid that encodes the protein in a translation system, the nucleic acid comprising a selector codon, and the translation system comprising an orthogonal tRNA (O-tRNA) that
recognizes the selector codon, an unnatural amino acid comprising a spectroscopic label, and an orthogonal aminoacyl tRNA synthetase (O-RS) that preferentially aminoacylates the O-tRNA with the unnatural amino acid,
wherein the unnatural amino acid comprises:
a) an isotopically labeled unnatural amino acid comprising an NMR active isotope selected from the group consisting of: ⁷Li, ¹³B, ¹⁴N, ¹⁵N, ¹⁷O, ¹⁹F, ²³Na, ²⁷Al, ²⁹Si, ³¹P, ⁵⁹Co, ⁷⁷Se, ¹¹³Cd, ¹¹⁹Sn, ¹⁹⁵Pt, d a combination thereof,
b) a spin-labeled amino acid, or
c) a chelator for a paramagnetic metal, thereby producing the spectroscopically labeled protein; and
subjecting the spectroscopically labeled protein to a spectroscopic technique, wherein the spectroscopic technique is NMR spectroscopy.

2. The method of claim 1, wherein the unnatural amino acid comprises an isotopically labeled unnatural amino acid, wherein the NMR active isotope is part of a methyl group, an amino group, an azido group, a keto group, a carboxy group, a cyano group, an alkyl group, an alkoxy group, an alkynyl moiety, a thiol group, a halogen atom, an aryl group, a sugar residue, a photocrosslinking moiety, or a photolabile group.

3. The method of claim 1, wherein the unnatural amino acid comprises an isotopically labeled unnatural amino acid, wherein the isotopically labeled unnatural amino acid comprises O-methyl-L-tyrosine.

4. The method of claim 3, wherein the isotopically labeled unnatural amino acid comprises ¹⁵N-labeled p-methoxyphenylalanine.

5. The method of claim 1, wherein the unnatural amino acid comprises an isotopically labeled unnatural amino acid, wherein the spectroscopically labeled protein further comprises a second isotopically labeled amino acid comprising a second NMR active isotope.

6. The method of claim 1, wherein the unnatural amino acid comprises a spin-labeled amino acid, wherein the spin-labeled amino acid comprises a nitroxide radical.

7. The method of claim 1, wherein the unnatural amino acid comprises a chelator for a paramagnetic metal, wherein the chelator comprises EDTA and the paramagnetic metal is selected from the group consisting of: Mn²⁺, Cu²⁺, Zn²⁺, Co²⁺, and Gd³⁺.

8. The method of claim 1, wherein the translation system comprises a cell.

9. The method of claim 8, wherein the cell comprises a prokaryotic cell.

10. The method of claim 8, wherein the cell comprises a eukaryotic cell.

11. The method of claim 10, wherein the eukaryotic cell is a yeast cell.

12. The method of claim 10, wherein the eukaryotic cell is a mammalian cell.

13. The method of claim 8, wherein the cell comprises an E. coli cell, and the O-tRNA and the O-RS comprise an M. jannaschii tyrosyl tRNA/tRNA synthetase pair.

14. The method of claim 8, wherein the cell comprises a eukaryotic cell, and wherein the O-tRNA and O-RS comprise a prokaryotic orthogonal tRNA/tRNA synthetase pair.

15. The method of claim 8, wherein the O-tRNA is from the same organism as the O-RS.

16. The method of claim 8, wherein the O-tRNA is not from the same organism as the O-RS.

17. The method of claim 1, wherein the spectroscopic technique is selected from the group consisting of: an HSQC experiment, a TROSY experiment, a SEA-TROSY experiment, a TROSY-HSQC experiment, a NOESY experiment, or an HSQC-NOESY experiment.

18. The method of claim 1, wherein the spectroscopically labeled protein comprises a ¹⁵N isotope, and wherein the spectroscopic technique comprises a solvent-exposed amine transverse relaxation optimized spectroscopy (SEA-TROSY) experiment.

19. The method of claim 1, wherein the spectroscopic technique is performed on the spectroscopically labeled protein in vivo.

20. The method of claim 1, wherein the subjecting step further comprises generating information regarding one or more changes in structure or dynamics of the spectroscopically labeled protein.

21. The method of claim 1, further comprising:
analyzing an interaction between the spectroscopically labeled protein and a ligand or substrate.

22. The method of claim 21, wherein the interaction comprises a change in conformation in the spectroscopically labeled protein.

23. The method of claim 21, wherein the interaction comprises a catalytic reaction performed by the spectroscopically labeled protein.

24. A method for assigning NMR resonances to one or more amino acid residues in a protein of interest, the method comprising:
providing an unnatural amino acid comprising an NMR active isotope selected from the group consisting of: ⁷Li, ¹³B, ¹⁴N, ¹⁵N, ¹⁷O, ¹⁹F, ²³Na, ²⁷Al, ²⁹Si, ³¹P, ⁵⁹CO, ⁷⁷Se, ¹¹³Cd, ¹¹⁹Sn, ¹⁹⁵Pt, and a combination thereof;
incorporating the unnatural amino acid and producing an isotopically-labeled protein of interest in a translation system comprising:
a) a nucleic acid encoding the protein of interest and comprising at least one selector codon for incorporating the unnatural amino acid at a specific site in the protein;
b) an orthogonal tRNA (O-tRNA) that recognizes the selector codon; and,
c) an orthogonal aminoacyl tRNA synthetase (O-RS) that preferentially aminoacylates the O-tRNA with the unnatural amino acid;
performing an NMR experiment on the isotopically labeled protein; and,
analyzing data generated due to an interaction between the NMR active isotope of the unnatural amino acid and a proximal atom, thereby assigning one or more NMR resonances to one or more amino acid residues in the protein.

25. The method of claim 24, wherein the NMR active isotope comprises ¹⁵N.

26. The method of claim 24, wherein the NMR experiment is selected from the group consisting of: an HSQC experiment, a TROSY experiment, a SEA-TROSY experiment, a TROSY-HSQC experiment, a NOESY experiment, and an HSQC-NOESY experiment.

27. The method of claim 24, wherein the specific site of the unnatural amino acid comprises an active site or ligand binding site of the protein.

28. The method of claim 24, wherein the specific site of the unnatural amino acid comprises a site proximal to an active site or ligand binding site of the protein.

29. The method of claim 24, wherein the translation system comprises a cell.

30. The method of claim 29, wherein performing the NMR experiment on the isotopically labeled protein comprises collecting data on a cellular extract comprising the isotopically labeled protein.

31. The method of claim 29, wherein performing the NMR experiment on the isotopically labeled protein comprises collecting data in vivo on the isotopically labeled protein.

32. A method for assigning an NMR resonance to an amino acid residue occupying a specific position in a protein of interest, the method comprising:
providing a first sample comprising the protein, wherein, at the specific position, the protein comprises an amino acid residue comprising an NMR active isotope;
performing an NMR experiment on the first sample and collecting a first set of data;
providing a second sample comprising the protein, wherein the protein comprises, at the specific position, an unnatural amino acid lacking the NMR active isotope;
performing an NMR experiment on the second sample and collecting a second set of data; and
comparing the first and second sets of data, whereby a resonance present in the first set and not present in the second set is assigned to the amino acid residue at the specific position.

33. The method of claim 32, wherein the NMR active isotope comprises ¹⁵N, ¹³C, or ¹⁹F.

34. The method of claim 32, wherein providing the second sample comprises:
translating a nucleic acid that encodes the protein in a translation system,
the nucleic acid comprising a selector codon for incorporating the unnatural amino acid at the specific position in the protein, and
the translation system comprising an orthogonal tRNA (O-tRNA) that recognizes the selector codon, the unnatural amino acid lacking the NMR active label, and an orthogonal aminoacyl tRNA synthetase (O-RS) that preferentially aminoacylates the O-tRNA with the unnatural amino acid.

35. A method for producing and analyzing a spectroscopically labeled protein, the method comprising:
translating a nucleic acid that encodes the protein in a translation system,
the nucleic acid comprising a selector codon for incorporating an unnatural
amino acid at a specific position in the protein, and
the translation system comprising an orthogonal tRNA (O-tRNA) that recognizes the selector codon, the unnatural amino acid, and an orthogonal aminoacyl tRNA synthetase (O-RS) that preferentially aminoacylates the O-tRNA with the unnatural amino acid,
thereby producing a translated protein comprising the unnatural amino acid at the specific position;
attaching a spectroscopic label to the unnatural amino acid in the translated protein, thereby producing the spectroscopically labeled protein; and
subjecting the spectroscopically labeled protein to a spectroscopic technique, which spectroscopic technique is NMR spectroscopy.

36. The method of claim 35, wherein the unnatural amino acid comprises p-acetyl-L-phenylalanine, m-acetyl-L-phenylalanine, O-allyl-L-tyrosine, O-(2-propynyl)-L-tyrosine, p-ethylthiocarbonyl-L-phenylalanine, p-(3-oxobutanoyl)-L-phenylalanine, p-azido-L-phenylalanine, orp-benzoyl-L-phenylalanine.

37. The method of claim 35, wherein the spectroscopic label comprises an isotopic label.

38. The method of claim 37, wherein the isotopic label comprises an NMR active isotope.

39. The method of claim 35, wherein the spectroscopic label comprises a spin-label.

40. The method of claim 39, wherein the spin-label comprises a nitroxide radical.

41. The method of claim 39, wherein the spin-label comprises 2,2,6,6-tetramethyl-piperidine-1-oxyl (TEMPO) or 2,2,5,5-tetramethylpyrroline-1-oxyl.

42. The method of claim 39, wherein subjecting the spectroscopically labeled protein to a spectroscopic technique comprises performing an NMR experiment on the spectroscopically labeled protein and collecting a first set of data; the method comprising reducing the spectroscopically labeled protein to provide a reduced form of the spectroscopically labeled protein, and performing an NMR experiment on the reduced form of the spectroscopically labeled protein and collecting a second set of data.

43. The method of claim 35, wherein the spectroscopic label comprises a chelator for a paramagnetic metal.

44. The method of claim 43, wherein the chelator comprises EDTA and the paramagnetic metal is selected from the group consisting of: Mn²⁺, Cu²⁺, Zn²⁺, Co²⁺, and Gd³⁺.

45. The method of claim 43, wherein attaching the spectroscopic label to the unnatural amino acid comprises covalently attaching the chelator to the unnatural amino acid and associating the paramagnetic metal with the chelator.

46. The method of claim 35, wherein attaching the spectroscopic label to the unnatural amino acid comprises covalently attaching the spectroscopic label to the unnatural amino acid.

47. The method of claim 35, comprising purifying the translated protein prior to attaching the spectroscopic label to the unnatural amino acid.

48. The method of claim 35, wherein the translation system comprises a cell.

49. The method of claim 48, wherein the cell comprises a prokaryotic cell.

50. The method of claim 48, wherein the cell comprises a eukaryotic cell.

51. The method of claim 50, wherein the eukaryotic cell is a yeast cell.

52. The method of claim 50, wherein the eukaryotic cell is a mammalian cell.

53. The method of claim 48, wherein the cell comprises an E. coli cell, and the O-tRNA and the O-RS comprise an M. jannaschii tyrosyl tRNA/tRNA synthetase pair.

54. The method of claim 48, wherein the cell comprises a eukaryotic cell, and wherein the O-tRNA and O-RS comprise a prokaryotic orthogonal tRNA/tRNA synthetase pair.

55. The method of claim 35, wherein the subjecting step further comprises generating information regarding a three-dimensional structure of the spectroscopically labeled protein.

56. The method of claim 35, wherein the subjecting step further comprises generating information regarding one or more changes in structure or dynamics of the spectroscopically labeled protein.

57. The method of claim 35, further comprising analyzing an interaction between the spectroscopically labeled protein and a ligand or substrate.

58. The method of claim 57, wherein the interaction comprises a change in conformation in the spectroscopically labeled protein.
